(19) United States
(12) Patent Application Publication
(10) Pub. No.: US 2012/0014987 A1
(43) Pub. Date: Jan. 19, 2012

(54) DNA VACCINE FOR ALZHEIMER'S DISEASE

(76) Inventor: Yoh Matsumoto, Tokyo (JP)

(21) Appl. No.: 13/257,713

(22) PCT Filed: Mar. 26, 2010
(86) PCT No.: PCT/JP2010/055308

§ 371 Date: Sep. 20, 2011

(30) Foreign Application Priority Data
Mar. 26, 2009 (JP) 2009-075832

(51) Int. Cl. A61K 39/00; A61P 37/04; A61P 25/28; C12N 15/63

(52) U.S. Cl. 424/198.1; 435/320.1

(57) ABSTRACT

The present invention aims to provide a DNA vaccine for Alzheimer's disease.

The present invention provides a recombinant vector which comprises DNA encoding amyloid β and DNA encoding a Th2 cytokine, as well as a DNA vaccine for Alzheimer's disease which comprises this vector.

### TECHNICAL FIELD

The present invention relates to DNA vaccines for Alzheimer's disease.

### BACKGROUND ART

Alzheimer's disease (AD) is a disease which frequently occurs in middle-aged or older people and whose major symptom is cognitive impairment (including memory disorder, orientation disturbance, learning disorder, attention disorder, spatial cognitive impairment, problem-solving impairment, etc.). Symptoms of this disease progress over several years, during which there often appear problem behaviors such as persecutory delusion, hallucination, offensive language, violence, wandering, and unclean behavior. This causes unwanted effects not only on patients themselves, but also on those around them including their family members and physicians, nurses, therapists, etc.

Alzheimer's disease has three pathological features, i.e., senile plaques (Aβdeposition), neurofibrillary tangles (tau deposition), and neuronal loss. Many studies have indicated that Aβ deposition occurs prior to tau deposition and neuronal changes, and the “amyloid hypothesis” has become widely known, which states that prevention of Aβ deposition would allow some avoidance of the subsequent events, i.e., tau accumulation in neurons, loss of neurons and so on.

Based on the amyloid hypothesis, Aβ peptide vaccine therapy has been proposed as a radical therapy for Alzheimer's disease (Non-patent Document 1). This vaccine therapy is based on the experiments in mice, in which the mice were externally administered with the Aβ peptide together with an immune activator (adjuvant) to induce in vivo production of anti-Aβ antibody to thereby reduce Aβ accumulation in the brain.

However, clinical trials in humans have been discontinued because of side effect problems, such as meningoencephalitis developed in cases receiving real drugs.

Following these clinical trials, further development has proceeded in an attempt to design vaccine formulations which do not cause encephalitis. The Aβ peptide is known to induce Th1 responses primarily through its C-terminal fragment and Th2 responses through its N-terminal fragment. Thus, clinical trials have been conducted for vaccines comprising an N-terminal fragment of Aβ attached to a carrier protein (Non-patent Document 2).

However, it was still difficult to overcome side effects including encephalitis.

For this reason, DNA vaccine therapy has been developed as a new vaccine therapy for Alzheimer's disease, which is an alternative to these Aβ peptide vaccines. Moreover, the inventors of the present invention have developed DNA vaccines which are effective for elimination of deposited Aβ and are also highly safe (Non-patent Document 3). A known example of the DNA vaccines developed by the inventors is an IgL-Aβ-Fc vaccine (Patent Document 1).

Recent studies have been indicating that there are various subspecies of neurotoxic Aβ. Aβ oligomers, which are most intensively analyzed, can be divided into two major types, i.e., those of low-molecular-weight type assembled from 2, 3 or 4 molecules and those of high-molecular-weight type assembled from 12 or more molecules. Moreover, strong neurotoxicity is also observed in pEAβ3-42, which is N-terminally truncated and pyrrole-derivatized by post-translational modification (Non-patent Documents 4 and 5). Furthermore, some findings are also being obtained, which suggest that other molecules such as ABri (Non-patent Document 6) and ADan, which have high amyloid aggregation propensity although their amino acid sequences are completely different from that of Aβ, are also involved in the onset of Alzheimer's disease.

### PRIOR ART DOCUMENTS

### Patent Documents

[Patent Document 1] WO 2005/014041 A2

### Non-Patent Documents

[Non-patent Document 1] Schenk D. et al., Nature 400: 173-177, 1999
[Non-patent Document 2] Masters C L et al., Brain 129: 2823-2839, 2006
[Non-patent Document 3] Y. Okura, et al., PNAS vol. 103: 9619-9624, 2006
[Non-patent Document 4] Saido et al., Neurosci Lett, 215, 173-176, 1996
[Non-patent Document 5] Schlenzig et al., Biochemistry, 48, 7072-7078, 2009
[Non-patent Document 6] Ghiso et al., Brain Pathol, 16, 71-79, 2006

### DISCLOSURE OF THE INVENTION

### Problems to be Solved by the Invention

In light of these previous studies, there has been a demand for the development of DNA vaccine therapy which is more effective and safer than existing DNA vaccines.

### Means for Solving the Problems

As a result of extensive and intensive efforts made to solve the above problems, the inventors of the present invention have constructed a vector which carries DNA encoding Aβ and DNA encoding a Th2 cytokine, as well as a vector which carries DNA encoding Aβ, DNA encoding a Th2 cytokine and DNA encoding an immunoglobulin Fc sequence, and have found that these vectors have a therapeutic effect when administered to model animals of Alzheimer's disease. The inventors have further found that the above vectors also induce antibodies against a wide variety of molecules with neurotoxicity and high amyloid aggregation propensity (e.g., pEAβ3-42, ABri, ADan). These findings led to the completion of the present invention.

Namely, the present invention is as follows.

(1) A recombinant vector, which comprises DNA encoding amyloid β and DNA encoding a Th2 cytokine.
(2) The vector according to (1) above, which further comprises DNA encoding an immunoglobulin Fc sequence.
(3) The vector according to (1) or (2) above, wherein the DNA encoding amyloid β consists of repeats of itself.
(4) The vector according to any one of (1) to (3) above, wherein the amyloid β is Aβ1-42.
(5) The vector according to any one of (1) to (4) above, wherein the Th2 cytokine is interleukin-4 or M-CSF.
(6) A DNA vaccine for prevention or treatment of Alzheimer's disease, which comprises the recombinant vector according to any one of (1) to (5) above.
(7) A DNA vaccine for elimination of brain Aβ, which comprises the recombinant vector according to any one of (1) to (5) above.
(8) An inducer of anti-Aβ antibody, which comprises the recombinant vector according to any one of (1) to (5) above.

### Advantageous Effect of the Invention

The present invention provides DNA vaccines for Alzheimer's disease. The DNA vaccines of the present invention can be regarded as having a high therapeutic effect on Alzheimer's disease, due to their high affinity for senile plaques. Moreover, the present invention allows induction of antibodies against a wide variety of molecules with neurotoxicity and high amyloid aggregation propensity (e.g., pEAβ3-42, ABri, ADan).

### BRIEF DESCRIPTION OF THE DRAWINGS

FIG. 1 shows the construction of a vector for use as the DNA vaccine of the present invention.

FIG. 2 shows the construction of a vector for use as the DNA vaccine of the present invention.

FIG. 3 shows the Aβ-eliminating effect produced by the DNA vaccine of the present invention upon administration to Alzheimer's disease model mice.

FIG. 4 shows increased levels of antibody with high affinity for Aβ deposition.

FIG. 5 shows the construction of a vector for use as the DNA vaccine of the present invention.

FIG. 6 shows the construction of a vector for use as the DNA vaccine of the present invention.

FIG. 7 shows the construction of a vector for use as the DNA vaccine of the present invention.

FIG. 8 shows the construction of a vector for use as the DNA vaccine of the present invention.

FIG. 9 shows the construction of a vector for use as the DNA vaccine of the present invention.

FIG. 10 shows the construction of a vector for use as the DNA vaccine of the present invention.

FIG. 11 shows the results obtained for the amount of Aβ produced in cultured cells transfected with the vaccine of the present invention and the amount of Aβ released into the extracellular environment.

FIG. 12 shows the anti-Aβ antibody-inducing effect in plasma produced by the vaccine of the present invention.

FIG. 13 shows the brain Aβ-eliminating effect produced by the vaccine of the present invention.

FIG. 14 shows the schedule of DNA vaccination and procedures for antibody titer assay.

FIG. 15 shows the anti-Aβ1-42 antibody-inducing effect produced by YM3711 in rabbits.

FIG. 16 shows the time course of anti-Aβ1-42 antibody induction mediated by YM3711 in rabbits.

FIG. 17 shows the anti-Aβ1-42 antibody-inducing effect produced by YM3711 in wild-type mice and in disease model mice.

FIG. 18 shows the anti-Aβ1-42 antibody-inducing effect produced by YM3711 in monkeys.

FIG. 19 shows the time course of anti-Aβ1-42 antibody induction in monkeys after immunization with YM3711.

FIG. 20 shows the anti-pEAb3-42 antibody-inducing effect produced by YM3711 in rabbits.

FIG. 21 shows the anti-ABri antibody-inducing effect produced by YM3711 in rabbits.

FIG. 22 shows the anti-ADan antibody-inducing effect produced by YM3711 in rabbits.

FIG. 23 shows the anti-pEAb3-42 antibody-inducing effect produced by YM3711 in monkeys.

FIG. 24 shows the anti-ADan antibody-inducing effect produced by YM3711 in monkeys.

### BEST MODES FOR CARRYING OUT THE INVENTION

The present invention will be described in more detail below. The following embodiments are illustrated to describe the present invention, and it is not intended to limit the present invention only to these embodiments. The present invention can be implemented in various modes, without departing from the spirit of the present invention. Moreover, this specification incorporates the contents disclosed in the specification and drawings of the Japanese patent application filed on Mar. 26, 2009 (Japanese Patent Application No. 2009-75832), based on which the present application claims priority.

The present invention is directed to a DNA vaccine for Alzheimer's disease. The present invention is also directed to a vector constructed by ligating the Aβ gene with the Th2 cytokine gene. The present invention is further directed to a vector constructed by ligating the above vector with an immunoglobulin Fc (IgFc) sequence.

Such a DNA vaccine is intended to cause Aβ protein production in cells into which the vaccine is incorporated, which is accomplished when a gene for Aβ protein production is integrated into a vector (plasmid or virus) for gene transfer and administered to a patient. Aβ thus produced stimulates the immune system and induces anti-Aβ antibody (Y. Okura, et al., BRAIN and NERVE 60(8): 931-940, 2008). DNA vaccines remain in the body for a long time after administration and continue to slowly produce the encoded peptides, so that excessive immune responses can be avoided, and DNA vaccines can also be modified due to their simple structure (Tang D C et al., Nature 356: 152-154, 1992; Barry M A et al., Nature 377: 632-635, 1995). Moreover, they are advantageous in that immune responses induced in a host are of Th2 type (Tang D C et al., Nature 356: 152-154, 1992; Ulmer J B et al., Science 259: 1745-1749, 1993; Hoffman S L et al., Ann N Y Acad Sci 772: 88-94, 1995).

In view of these facts, the inventors of the present invention have conceived that a gene encoding a Th2 cytokine is introduced together with the Aβ gene to thereby suppress side effects caused by cellular immunity, and have also constructed actual vaccines for Alzheimer's disease to study their effects. As a result, the vector of the present invention showed a high Aβ-eliminating effect at low doses and at reduced frequency. Moreover, the inventors have succeeded in promoting the extracellular release of expressed Aβ to more strongly stimulate immune responses against Ap by introduction of a gene encoding an IgFc sequence, in addition to the genes encoding Aβ and a Th2 cytokine. Further, the inventors have succeeded in developing a vaccine with a higher brain Aβ-eliminating effect by construction of a vector comprising repeats of Aβ-encoding DNA. Furthermore, the inventors have found that the above vector also induces antibodies against a wide variety of molecules with neurotoxicity and high amyloid aggregation propensity (e.g., pEAβ3-42, ABri, ADan).

Thus, the vector of the present invention comprises DNA encoding Aβ and DNA encoding a Th2 cytokine as foreign genes. In another embodiment, the vector of the present invention comprises DNA encoding an IgFc sequence, in addition to the DNA encoding Aβ and the DNA encoding a Th2 cytokine. In yet another embodiment, the DNA encoding Aβ contained in the vector of the present invention consists of repeats of itself.

Although such Aβ, Th2 cytokine and IgFc sequence may be derived from animals of the same or different species as the target animal to be administered with the DNA vaccine, those derived from animals of the same species are preferred for use.

DNA encoding Aβ, DNA encoding a Th2 cytokine and DNA encoding an IgFc sequence have already been cloned. Thus, DNAs contained in the vector of the present invention can be obtained by standard genetic engineering procedures. For example, it is possible to use nucleic acid synthesis techniques with a DNA synthesizer, which are commonly used as genetic engineering procedures. Alternatively, it is also possible to use PCR techniques in which DNA sequences serving as templates are isolated or synthesized, and primers specific for each DNA are then designed to amplify each gene sequence with a PCR system, or to use gene amplification techniques in which cloning vectors are used. The techniques listed above would be readily performed by those skilled in the art, according to Molecular cloning 2nd Edt. Cold Spring Harbor Laboratory Press (1989), etc. For purification of the resulting PCR products, any known method may be used.

Amyloid β protein (Aβ) is a polypeptide composed of 40 to 43 amino acids, which is cleaved from its precursor protein (APP: amyloid β protein precursor) by the action of β- and γ-secretases.

To present antigenicity against Aβ, such a polypeptide comprises 15 or more contiguous amino acids, preferably 20 or more contiguous amino acids derived from the native Aβ amino acid sequence. A polypeptide comprising the native full-length Aβ may also be used.

The nucleotide sequences of these DNAs are available from a certain database. For example, Genbank Accession No. NC_{—}000021.7 may be used for human Aβ, while Accession No. NC_{—}000082.5 may be used for mouse Aβ.

In a preferred embodiment of the present invention, the above DNAs may be used as templates for PCR with primers specific for these genes to prepare each DNA region with each fragment length. In the present invention, it is possible to use a polypeptide of a region covering 43 amino acids cleaved by the action of γ-secretase (referred to as “Aβ1-43”), a region covering N-terminal Aβ amino acids 1 to 20 (referred to as “Aβ1-20”), a region covering N-terminal amino acids 1 to 40 (referred to as “Aβ1-40”), or a region covering N-terminal amino acids 1 to 42 (referred to as “Aβ1-42”). The amino acid sequences of human Aβ1-43, Aβ1-20, Aβ1-40 and Aβ1-42 are shown in SEQ ID NOs: 2, 4, 6 and 8, respectively, while those of mouse Aβ1-43, Aβ1-20, Aβ1-40 and Aβ1-42 are shown in SEQ ID NOs: 10, 12, 14 and 16, respectively.

The nucleotide sequences of DNAs encoding human Aβ1-43, Aβ1-20, Aβ1-40 and Aβ1-42 are shown in SEQ ID NOs: 1, 3, 5 and 7, respectively, while the nucleotide sequences of DNAs encoding mouse Aβ1-43, Aβ1-20, Aβ1-40 and Aβ1-42 are shown in SEQ ID NOs: 9, 11, 13 and 15, respectively.

DNAs encoding human or mouse Aβ1-20, Aβ1-40 and Aβ1-42 may be prepared by PCR or other techniques from DNA encoding human or mouse Aβ1-43.

In addition to the above DNAs encoding human or mouse Aβ1-43, Aβ1-20, Aβ1-40 and Aβ1-42, the following DNAs may also be used in the present invention.

DNA which is hybridizable under stringent conditions with DNA consisting of a nucleotide sequence complementary to the nucleotide sequence shown in SEQ ID NO: 1 and which encodes a protein having human Aβ activity.

DNA which is hybridizable under stringent conditions with DNA consisting of a nucleotide sequence complementary to the nucleotide sequence shown in SEQ ID NO: 3 and which encodes a protein having human Aβ activity.

DNA which is hybridizable under stringent conditions with DNA consisting of a nucleotide sequence complementary to the nucleotide sequence shown in SEQ ID NO: 5 and which encodes a protein having human Aβ activity.

DNA which is hybridizable under stringent conditions with DNA consisting of a nucleotide sequence complementary to the nucleotide sequence shown in SEQ ID NO: 7 and which encodes a protein having human Aβ activity.

DNA which is hybridizable under stringent conditions with DNA consisting of a nucleotide sequence complementary to the nucleotide sequence shown in SEQ ID NO: 9 and which encodes a protein having mouse Aβ activity.

DNA which is hybridizable under stringent conditions with DNA consisting of a nucleotide sequence complementary to the nucleotide sequence shown in SEQ ID NO: 11 and which encodes a protein having mouse Aβ activity.

DNA which is hybridizable under stringent conditions with DNA consisting of a nucleotide sequence complementary to the nucleotide sequence shown in SEQ ID NO: 13 and which encodes a protein having mouse Aβ activity.

DNA which is hybridizable under stringent conditions with DNA consisting of a nucleotide sequence complementary to the nucleotide sequence shown in SEQ ID NO: 15 and which encodes a protein having mouse Aβ activity.

In the context of the present invention, the term “Aβ activity” is intended to mean the ability to produce, accumulate and/or aggregate Aβ in the brain of a subject (e.g., human, mouse) to thereby form Aβ deposition (senile plaques). Aβ activity can be measured by immunological procedures such as immunohistological staining, ELISA and the like. For example, in the case of immunohistological staining, a protein to be evaluated may be expressed in the brain of a subject animal (e.g., mouse), and sections of the expressed tissue may be immunostained with anti-Aβ antibody to detect Aβ production, accumulation, aggregation and/or deposition for activity assay.

Human Aβ1-43, human Aβ1-20, human Aβ1-40, human Aβ1-42, mouse Aβ1-43, mouse Aβ1-20, mouse Aβ1-40 and mouse Aβ1-42 have their own Aβ activity. Thus, for example, in the case of a protein expressed from DNA which is hybridizable under stringent conditions with DNA consisting of a nucleotide sequence complementary to the nucleotide sequence shown in SEQ ID NO: 1 and which encodes a protein having human Aβ activity, it is sufficient for this protein to have Aβ activity equal to that of human Aβ1-43. The same also applies to proteins expressed from DNAs which are hybridizable under stringent conditions with DNAs consisting of nucleotide sequences complementary to the nucleotide sequences shown in the other SEQ ID NOs and which encode proteins having Aβ activity.

Stringent conditions in the above hybridization include, for example, conditions of 0.1×SSC to 10×SSC, 0.1% to 1.0% SDS and 20° C. to 80° C., more specifically prehybridization at 37° C. to 56° C. for 30 minutes or longer and the subsequent one to three washings in 0.1×SSC, 0.1% SDS at room temperature for 10 to 20 minutes. For detailed procedures of hybridization, reference may be made to “Molecular Cloning, A Laboratory Manual 2nd ed.” (Cold Spring Harbor Press (1989)), etc.

It is also possible to use DNA which shares a homology of 50% or more, 60% or more, 70% or more, 80% or more, 90% or more, 95% or more, 98% or more, or 99% or more with the nucleotide sequence shown in SEQ ID NO: 1, 3, 5 or 7 and which encodes a protein functionally equivalent to the structural protein of human Aβ1-43, Aβ1-20, Aβ1-40 or Aβ1-42.

It is further possible to use DNA which shares a homology of 50% or more, 60% or more, 70% or more, 80% or more, 90% or more, 95% or more, 98% or more, or 99% or more with the nucleotide sequence shown in SEQ ID NO: 9, 11, 13 or 15 and which encodes a protein functionally equivalent to the structural protein of mouse Aβ1-43, Aβ1-20, Aβ1-40 or Aβ1-42.

The term “functionally equivalent” is intended to mean having the function of causing Aβ deposition (senile plaques) in tissues, etc.

In the present invention, DNA encoding Aβ may consists of repeats of the DNA encoding Aβ. A vector comprising repeats of Aβ-encoding DNA allows multiple Aβ molecules to be expressed simultaneously. Moreover, Aβ molecules released into the extracellular environment would form an oligomer. This oligomer stimulates the immune system to induce anti-Aβ oligomer antibody. As a result, it can be expected to eliminate Aβ oligomers whose neurotoxicity is stronger than that of Aβ monomers.

There is no limitation on the repeated number of Aβ-encoding DNAs as long as the folded structure of Aβ is formed to give higher antigenicity than that of Aβ monomers. The number of repeats preferably ranges from 2 to 4, more preferably from 3 to 4.

In the context of the present invention, the term “Th2” is intended to mean a subgroup of CD4⁺ T cells (commonly called helper T cells), and refers to cells that are induced to differentiate from T cells not experiencing any contact with antigen proteins (i.e., naive T cells) upon stimulation with cytokines such as IL-4 or IL-13. Th2 is involved in regulation of humoral immunity, and cytokines produced from Th2 promote antibody production. Examples of Th2 cytokines include IL-4 (interleukin-4), IL-5, IL-6, IL-10, IL-13, M-CSF (macrophage-colony-stimulating factor) and so on, with IL-4 being preferred.

Genbank Accession Nos. NM_{—}000589.2 and NM_{—}021283.1 may be used for human IL-4 and mouse IL-4, respectively. Likewise, Genbank Accession Nos. NM_{—}000757.4 and NM_{—}007778.3 may be used for human M-CSF and mouse M-CSF, respectively.

SEQ ID NOs of the nucleotide sequences of the above DNAs encoding IL-4 and M-CSF are shown below.

Human IL-4: SEQ ID NO: 17

Mouse IL-4: SEQ ID NO: 18

Human M-CSF: SEQ ID NO: 19

Mouse M-CSF: SEQ ID NO: 20

In addition to the above DNAs encoding Th2 cytokines, the following DNAs may also be used in the present invention.

DNA which is hybridizable under stringent conditions with DNA consisting of a nucleotide sequence complementary to the nucleotide sequence shown in SEQ ID NO: 17 and which encodes a protein having human IL-4 activity.

DNA which is hybridizable under stringent conditions with DNA consisting of a nucleotide sequence complementary to the nucleotide sequence shown in SEQ ID NO: 18 and which encodes a protein having mouse IL-4 activity.

DNA which is hybridizable under stringent conditions with DNA consisting of a nucleotide sequence complementary to the nucleotide sequence shown in SEQ ID NO: 19 and which encodes a protein having human M-CSF activity.

DNA which is hybridizable under stringent conditions with DNA consisting of a nucleotide sequence complementary to the nucleotide sequence shown in SEQ ID NO: 20 and which encodes a protein having mouse M-CSF activity.

The term “IL-4 activity” is intended to mean the ability to promote Th2 cell differentiation, immunoglobulin class switch, and antibody production in B cells. IL-4 activity can be measured, for example, by immunological procedures such as ELISA, EIA and the like.

The term “M-CSF activity” is intended to mean the ability to promote differentiation and proliferation of monocytes, macrophages and other cells. M-CSF activity can be measured, for example, by immunological procedures such as ELISA, EIA and the like.

Moreover, it is also possible to use DNA which shares a homology of 50% or more, 60% or more, 70% or more, 80% or more, 90% or more, 95% or more, 98% or more, or 99% or more with the nucleotide sequence shown in SEQ ID NO: 17 and which encodes a protein functionally equivalent to the structural protein of human IL-4.

Further, it is also possible to use DNA which shares a homology of 50% or more, 60% or more, 70% or more, 80% or more, 90% or more, 95% or more, 98% or more, or 99% or more with the nucleotide sequence shown in SEQ ID NO: 18 and which encodes a protein functionally equivalent to the structural protein of mouse IL-4.

Furthermore, it is also possible to use DNA which shares a homology of 50% or more, 60% or more, 70% or more, 80% or more, 90% or more, 95% or more, 98% or more, or 99% or more with the nucleotide sequence shown in SEQ ID NO: 19 and which encodes a protein functionally equivalent to the structural protein of human M-CSF.

Furthermore, it is also possible to use DNA which shares a homology of 50% or more, 60% or more, 70% or more, 80% or more, 90% or more, 95% or more, 98% or more, or 99% or more with the nucleotide sequence shown in SEQ ID NO: 20 and which encodes a protein functionally equivalent to the structural protein of mouse M-CSF.

The immunoglobulin Fc (IgFc) sequence to be used in the present invention refers to a C-terminal fragment of the H chain, which is among the fragments obtained by papain digestion of immunoglobulin molecules.

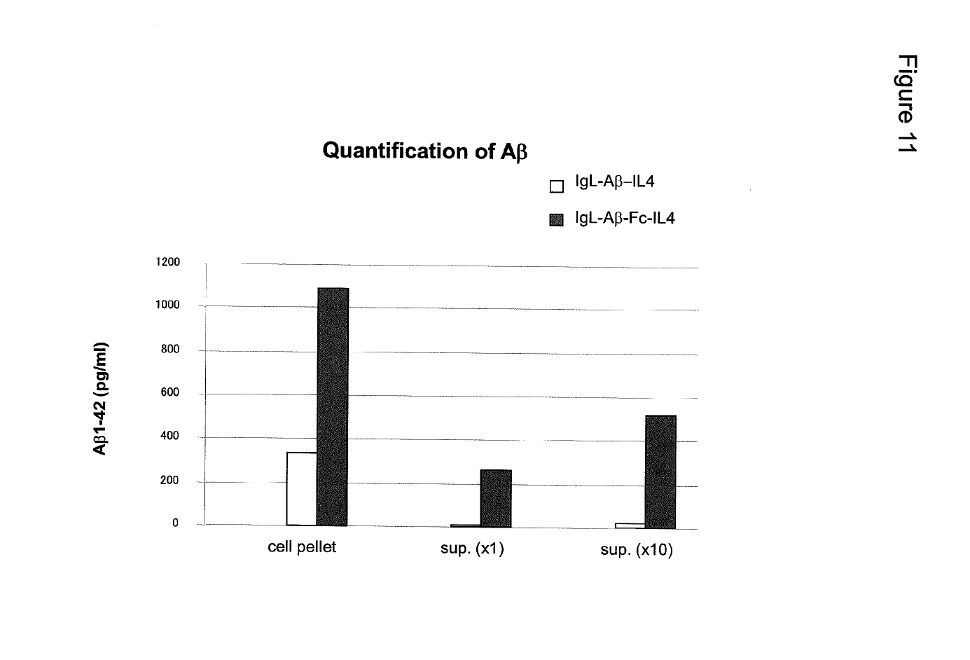

In the present invention, a vector comprising DNA encoding an IgFc sequence allows addition of the IgFc sequence to Aβ to thereby promote intracellular transcription and translation of Aβ. Moreover, such a vector also enhances release of the expressed Aβ into the extracellular environment, so that the immune system can be stimulated more strongly (see FIG. 11).

Examples of IgFc sequences to be used in the present invention include human and mouse IgFc sequences. With respect to the nucleotide sequences of DNAs encoding human and mouse IgFc sequences, Genbank Accession Nos. BC014258 and XM_{—}484178.3 may be used for human and mouse IgFc sequences, respectively.

SEQ ID NOs of the nucleotide sequences of DNAs encoding human and mouse IgFc sequences to be used in the present invention are shown below.

Human IgFc sequence: SEQ ID NO: 32

Mouse IgFc sequence: SEQ ID NO: 33

The nucleotide sequences shown in SEQ ID NOs: 32 and 33 each have a mutation to replace cysteine residues contained in the original IgFc sequence with serine residues. This is intended to avoid the formation of disulfide linkages.

Moreover, the following DNAs may also be used as DNAs encoding human and mouse IgFc sequences to be used in the present invention.

DNA which is hybridizable under stringent conditions with DNA consisting of a nucleotide sequence complementary to the nucleotide sequence shown in SEQ ID NO: 32 and which encodes a protein having human IgFc activity.

DNA which is hybridizable under stringent conditions with DNA consisting of a nucleotide sequence complementary to the nucleotide sequence shown in SEQ ID NO: 33 and which encodes a protein having mouse IgFc activity.

In the context of the present invention, the term “IgFc activity” is intended to mean the ability to promote intracellular production and extracellular release of Aβ protein. For example, the IgFc activity of a certain protein may be measured as follows: a fusion protein between this protein and Aβ protein is expressed in cultured cells, and an increase in the amount of Aβ protein present in the cultured cells or in the culture supernatant is determined. Quantification of Aβ protein may be accomplished by using immunological procedures such as ELISA, EIA and the like.

Moreover, in the present invention, it is also possible to use DNA which shares a homology of 50% or more, 60% or more, 70% or more, 80% or more, 90% or more, 95% or more, 98% or more, or 99% or more with the nucleotide sequence shown in SEQ ID NO: 32 and which encodes a protein having human IgFc activity.

Further, it is also possible to use DNA which shares a homology of 50% or more, 60% or more, 70% or more, 80% or more, 90% or more, 95% or more, 98% or more, or 99% or more with the nucleotide sequence shown in SEQ ID NO: 33 and which encodes a protein having mouse IgFc activity.

IgFc activity is as defined above.

The above DNAs may be obtained by chemical synthesis or may be obtained from cDNA and genomic libraries by known hybridization techniques (e.g., colony hybridization, plaque hybridization, Southern blotting) using DNA consisting of the nucleotide sequence shown in SEQ ID NO: 1, 3, 5, 7, 9, 11, 13, 15, 17, 18, 19, 20, 32 or 33 or a fragment thereof as a probe.

To construct recombinant vectors, standard genetic engineering procedures may be used.

For example, samples of DNA encoding Aβ of interest, DNA encoding a Th2 cytokine and DNA encoding an IgFc sequence are prepared by PCR or other techniques. PCR may be accomplished in a standard manner by using Taq polymerase or other DNA polymerases. The amplified fragments of interest are digested with restriction enzymes and then inserted into restriction enzyme sites or a multicloning site in a plasmid vector such as pBluescript™ (Stratagene). The resulting PCR products are confirmed for their nucleotide sequences with a sequencer to select a plasmid containing the proper sequence. It is preferred that such a DNA sample can be confirmed as an electrophoretically single plasmid.

As a promoter contained in the recombinant vector of the present invention, actin promoter, EF1 promoter, CMV promoter, CAG promoter or the like may be used. These promoters may each be ligated to an appropriate plasmid.

“Stringent conditions” are as defined above. The phrase “having promoter activity” is intended to mean having the ability to transcribe a gene encoding a structural protein or a non-structural protein.

In the vector of the present invention, the above Aβ and Th2 cytokine, and optionally the IgFc sequence are contained in operable form to ensure their expression. Namely, the transgenes (DNAs) are inserted into the vector in a mode which allows expression of the transgenes under the control of appropriate regulatory elements. The DNAs encoding Aβ, Th2 cytokine and IgFc sequence may be separately inserted into different sites in the same vector or may be inserted in tandem. In this context, regulatory elements refer to, for example, promoters, enhancers, transcription terminators and so on.

The vector of the present invention may carry an additional foreign gene at a position different from the region(s) into which the DNAs encoding Aβ, Th2 cytokine and IgFc sequence are inserted. Such a foreign gene may be, for example, but is not limited to, a marker gene for vector monitoring or a regulatory gene for the immune system, such as cytokines and hormones.

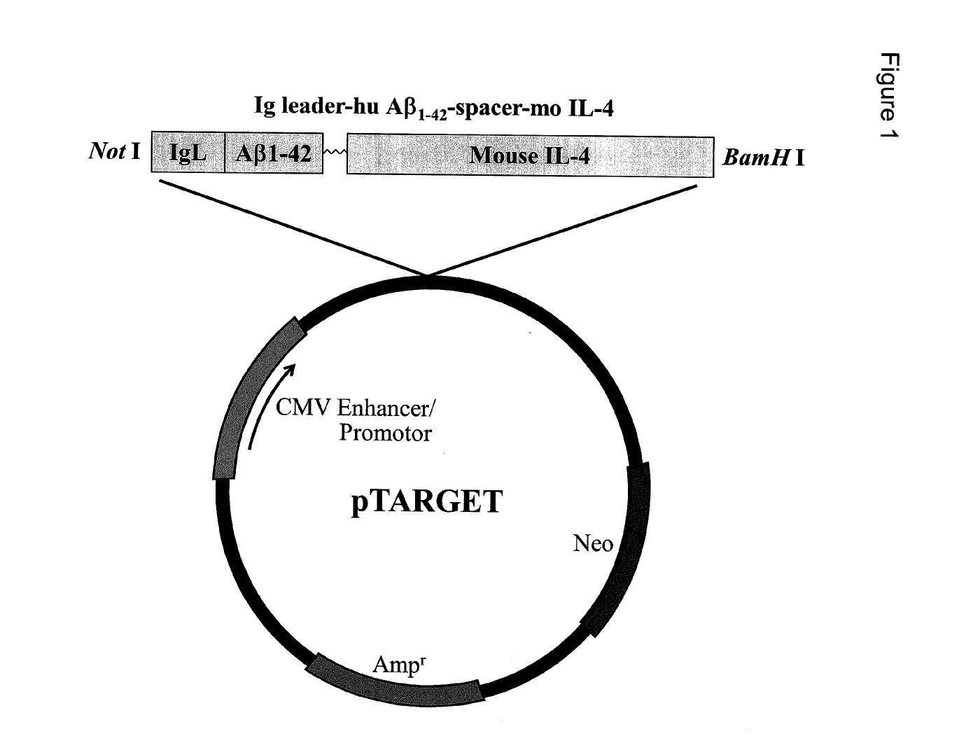

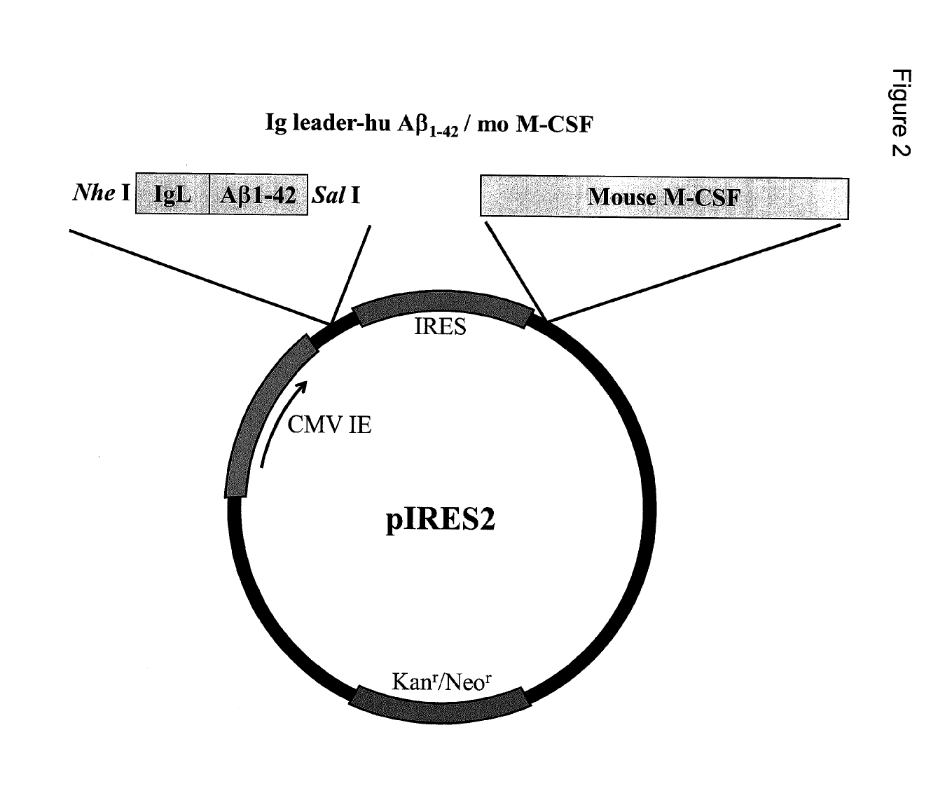

The vector of the present invention may be exemplified by pTARGET in which the Aβ1-42 gene and the IL-4 gene are inserted downstream of the CMV promoter (FIG. 1), as well as pIRES2 in which the Aβ1-42 gene and the M-CSF gene are inserted downstream of the CMV promoter (FIG. 2).

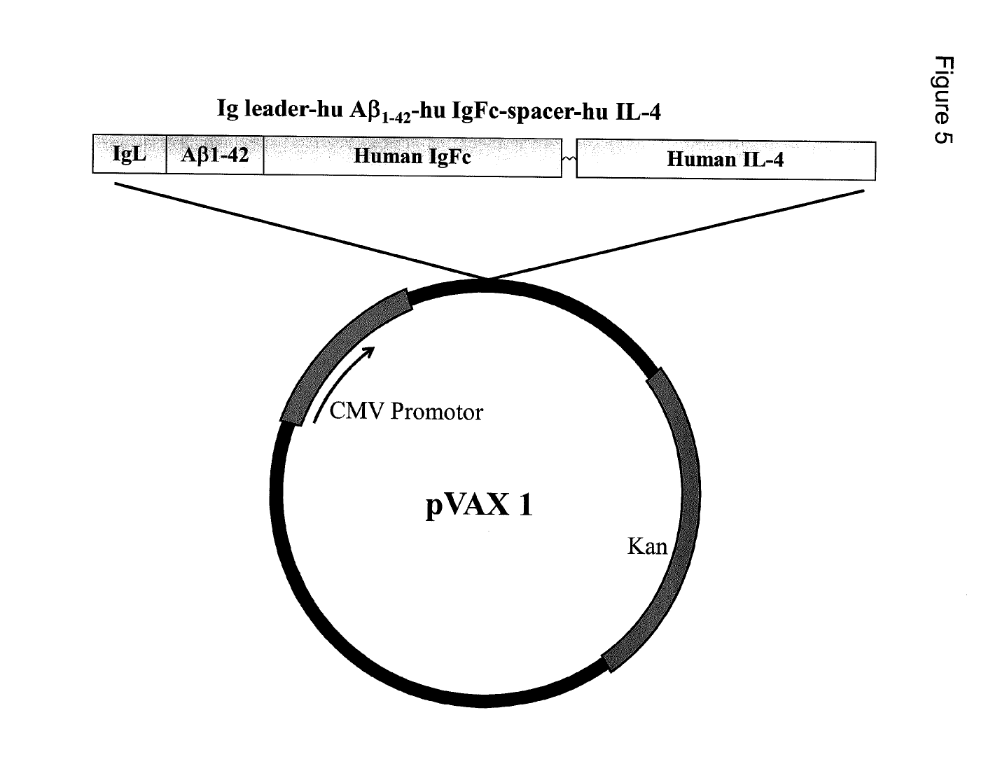

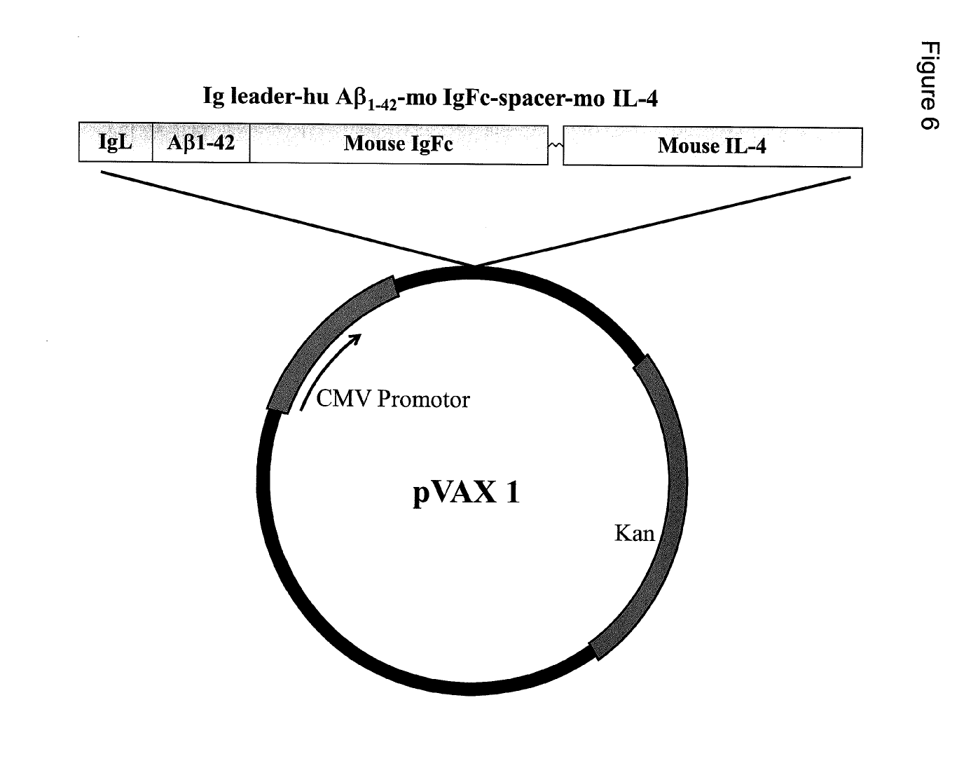

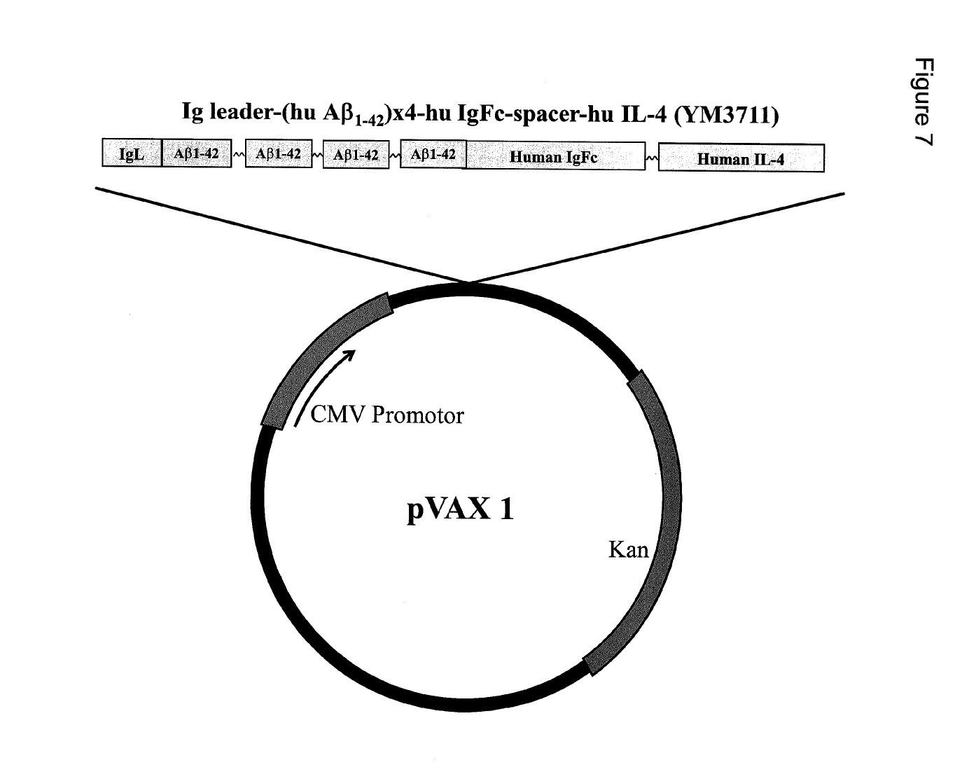

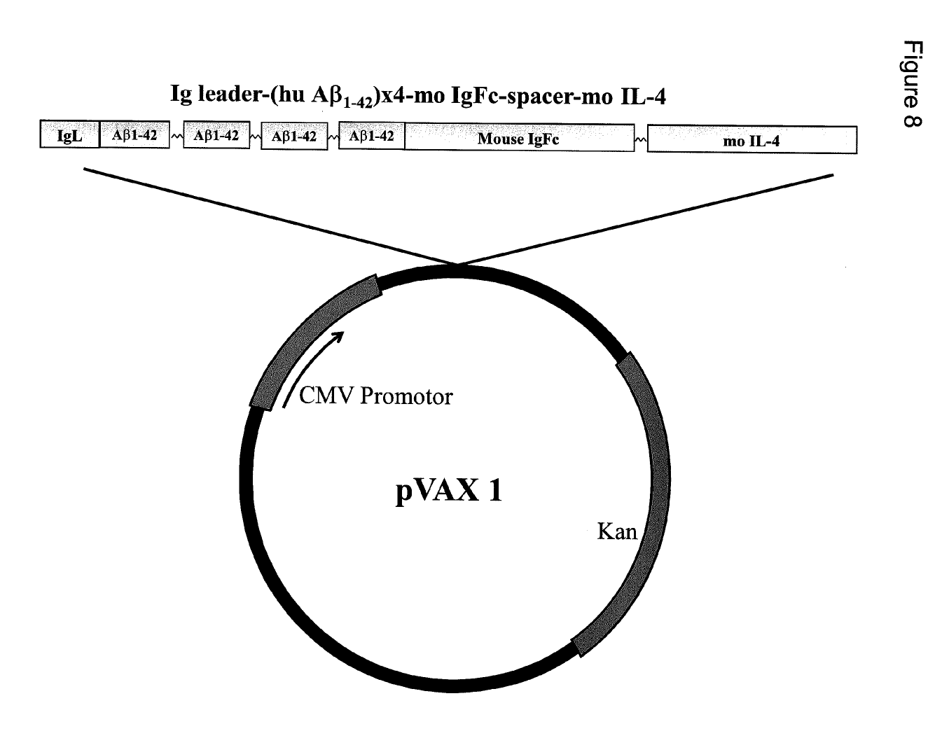

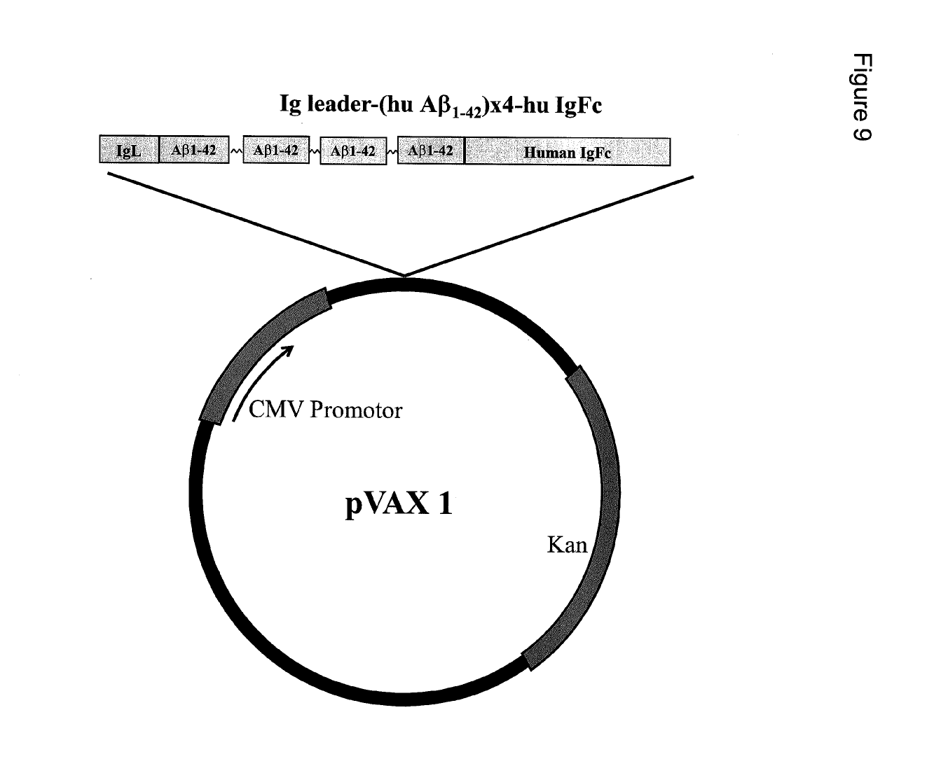

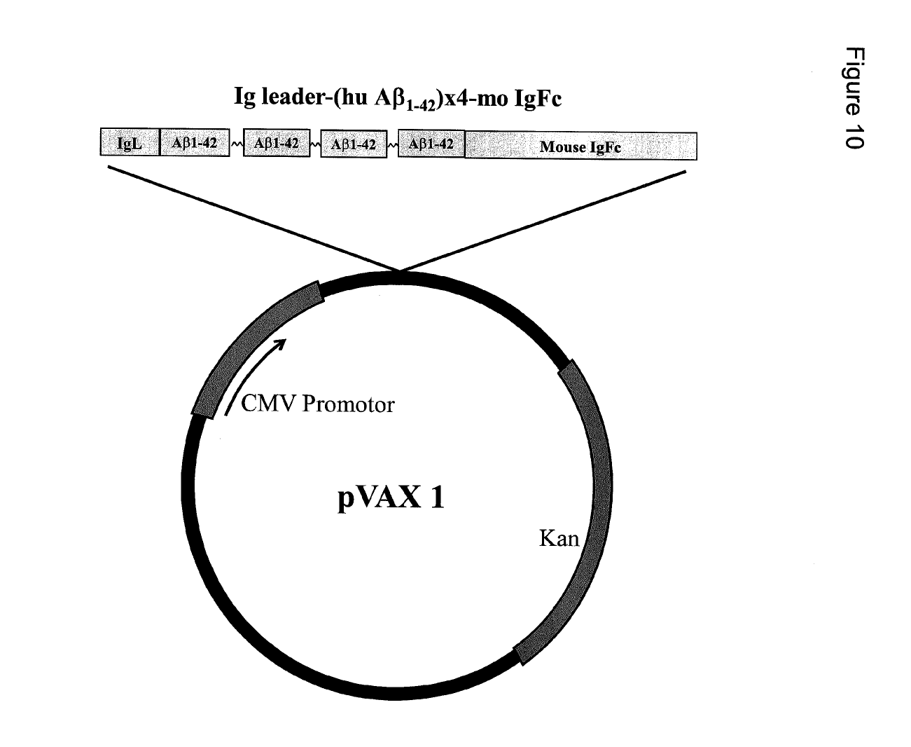

Moreover, the vector of the present invention may also be exemplified by pVAX1 in which the Aβ1-42 gene, the IgFc gene and the IL-4 gene are inserted downstream of the CMV promoter (FIGS. 5 and 6), pVAX1 in which repeats of the Aβ1-42 gene (DNA), the IgFc gene and the IL-4 gene are inserted downstream of the CMV promoter (FIGS. 7 and 8), as well as pVAX1 in which repeats of the Aβ1-42 gene and the IgFc gene are inserted downstream of the CMV promoter (FIGS. 9 and 10).

pTARGET, in which the Aβ1-42 gene and the IL-4 gene are inserted downstream of the CMV promoter, preferably comprises an Ig leader (IgL) sequence upstream of the Aβ1-42 gene and a spacer sequence between the Aβ1-42 gene and the IL-4 gene. More preferably, this pTARGET comprises the CMV promoter, the Ig leader sequence, the Aβ1-42 gene, the spacer sequence and the IL-4 gene in this order (FIG. 1).

Likewise, pIRES2, in which the Aβ1-42 gene and the M-CSF gene are inserted downstream of the CMV promoter, preferably comprises an Ig leader sequence upstream of the Aβ1-42 gene and an IRES sequence between the Aβ1-42 gene and the M-CSF gene. More preferably, this pIRES2 comprises the CMV promoter, the Ig leader sequence, the Aβ1-42 gene, the IRES sequence and the M-CSF gene in this order (FIG. 2).

pVAX1, in which the Aβ1-42 gene, the IgFc gene and the IL-4 gene are inserted downstream of the CMV promoter, preferably comprises an Ig leader sequence between the CMV promoter and the Aβ1-42 gene and a spacer sequence between the IgFc gene and the IL-4 gene. More preferably, this pVAX1 comprises the CMV promoter, the Ig leader sequence, the Aβ1-42 gene, the IgFc gene, the spacer sequence and the IL-4 gene in this order (FIGS. 5 and 6).

pVAX1, in which repeats of the Aβ1-42 gene, the IgFc gene and the IL-4 gene are inserted downstream of the CMV promoter, preferably comprises an Ig leader sequence between the CMV promoter and repeats of the Aβ1-42 gene, as well as spacer sequences between the individual Aβ1-42 genes and between the IgFc gene and the IL-4 gene. More preferably, this pVAX1 comprises the CMV promoter, the Ig leader sequence, repeats of the Aβ1-42 gene (containing spacer sequences between the individual Aβ1-42 genes), the IgFc gene, the spacer sequence and the IL-4 gene in this order (FIGS. 7 and 8).

pVAX1, in which repeats of the Aβ1-42 gene and the IgFc gene are inserted downstream of the CMV promoter, preferably comprises an Ig leader sequence between the CMV promoter and repeats of the Aβ1-42 gene, as well as spacer sequences between the individual Aβ1-42 genes. More preferably, this pVAX1 comprises the CMV promoter, the Ig leader sequence, repeats of the Aβ1-42 gene (containing spacer sequences between the individual Aβ1-42 genes) and the IgFc gene in this order (FIGS. 9 and 10).

The above vectors each have genes of mouse or human origin. The vectors having genes of mouse origin can be used in preclinical trials or reagents, while the vectors having genes of human origin can be used in pharmaceutical compositions or reagents.

Namely, the present invention provides a pharmaceutical composition (DNA vaccine) for prevention or treatment of Alzheimer's disease, which comprises the above vector. In another embodiment, the present invention provides a pharmaceutical composition (DNA vaccine) for elimination of brain Aβ, which comprises the above vector. In the context of the present invention, the term “elimination” or “eliminating” is intended to mean reducing the amount of Aβ present in the brain, which encompasses reducing the amount of brain Aβ accumulated, aggregated or deposited in the brain. Moreover, the pharmaceutical composition of the present invention, which comprises the above vector, may also be used as a DNA vaccine for suppressing an increase in brain Aβ levels.

In yet another embodiment, the present invention provides an inducer of anti-Aβ antibody, which comprises the above vector. In still yet another embodiment, the present invention provides an inducer of anti-pEAβ3-42 antibody, anti-ABri antibody or anti-ADan antibody, which comprises the above vector.

pEAβ3-42 is a molecule obtained from Aβ1-42 by N-terminal truncation with gulutaminyl cyclase (QC) and post-translational modification (pyrrole modification). pEAβ3-42 is highly neurotoxic and this molecule itself has a high tendency to aggregate. It also has the ability to enhance the aggregation propensity of unmodified Aβ, and is one of the major factors responsible for lesion development in Alzheimer's disease.

ABri is a causative molecule for familial British dementia, while ADan is a causative molecule for familial Danish dementia. ABri and ADan are molecules which are cleaved as long molecules from their precursor proteins due to a gene mutation in the stop codon region of each precursor protein. ABri and ADan have high amyloid aggregation propensity (amyloid is a generic name for events in which small molecules are aggregated and deposited) and are key molecules in disease progression. Thus, they appear to also play some role in the development of Alzheimer lesions.

In a case where the vector of the present invention is used as a DNA vaccine or as an inducer of anti-Aβ antibody, anti-pEAβ3-42 antibody, anti-ABri antibody or anti-ADan antibody (hereinafter also referred to as “inducers of anti-Aβ antibody and others”), gene transfer may be accomplished either by direct administration in which the vector is directly injected into a target site in the body or by indirect administration in which the vector is infected into patient's own cells or other cells for gene transfer, and the infected cells are then injected into a target site. For direct injection of the vector, intramuscular injection or the like may be used.

Alternatively, the vector of the present invention may also be administered by being introduced into phospholipid vesicles such as liposomes. The vesicles holding the vector of the present invention are introduced by lipofection into certain specific cells. Then, the resulting cells are administered systemically, for example, by the intravenous or intraarterial route. They may be administered locally to the brain, etc.

Examples of lipids used to form liposome structures include phospholipids, cholesterols and nitrogen-containing lipids. Commonly preferred are phospholipids, including natural phospholipids such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, and lysolecithin, as well as hydrogenated products thereof obtained in a standard manner. It is also possible to use synthetic phospholipids such as dicetyl phosphate, distearoylphosphatidylcholine, dipalmitoylphosphatidykholine, dipalmitoylphosphatidylethanolamine, dipalmitoylphosphatidylserine, eleostearoylphosphatidylcholine, eleostearoylphosphatidylethanolamine and so on.

The preparation of liposomes is not limited in any way as long as the resulting liposomes hold DNAs. Liposomes may be prepared in a conventional manner, for example, by reversed-phase evaporation, ether injection, surfactant-based techniques, etc.

Lipids including these phospholipids may be used either alone or in combination. In this case, it is also possible to increase the binding rate of electrically negative DNA when using a lipid whose molecule contains an atomic group having a cationic group (e.g., ethanolamine or choline). In addition to these major phospholipids used for liposome formation, it is also possible to use other additives such as cholesterols, stearyl amine, α-tocopherol and the like, which are generally known as liposome-forming additives. The liposomes thus obtained may further comprise a membrane fusion promoter (e.g., polyethylene glycol) in order to enhance their uptake into cells in the affected area or target tissue.

The vaccine of the present invention or the inducers of anti-Aβ antibody and others according to the present invention may be formulated in a routine manner and may comprise pharmaceutically acceptable carriers. Such carriers may be additives and include water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives, etc.

The above additives may be selected alone or in combination from among those listed above, depending on the dosage form of each therapeutic agent of the present invention. For example, for use as injectable formulations, the purified vector may be dissolved in a solvent (e.g., physiological saline, buffer, glucose solution) and then supplemented with Tween 80, Tween 20, gelatin, human serum albumin or the like. Alternatively, the ingredients may be lyophilized for use as dosage forms that are reconstituted before use. Examples of excipients for lyophilization include sugars such as mannitol, glucose, lactose, sucrose, mannitol and sorbitol; starches such as those derived from corn, wheat, rice, potato and other plants; celluloses such as methylcellulose, hydroxypropylmethylcellulose and carboxymethylcellulose sodium; gums such as gum arabic and gum tragacanth; as well as gelatin, collagen and so on.

Targets to be administered with the DNA vaccine of the present invention or with the inducers of anti-Aβ antibody and others according to the present invention include, for example, humans and all other mammals such as non-human primates (e.g., monkeys), rodents (e.g., mice and rats), rabbits, goats, sheep, pigs, cattle and dogs, with humans being more preferred. Animals serving as targets to be administered may be, for example, those suffering from Alzheimer's disease, those suspected to have Alzheimer's disease, those showing enhanced Aβ deposition, those showing enhanced tau deposition, or those showing neuronal loss.

The dosage of the DNA vaccine of the present invention or the inducers of anti-Aβ antibody and others according to the present invention will vary depending on the age, sex and symptoms of a patient, the route of administration, the frequency of administration, and the intended dosage form. The mode of administration is selected as appropriate for the age and symptoms of a patient. The effective dosage of the DNA vaccine is an amount of the vaccine required to reduce the signs or condition of the disease. The therapeutic effect and toxicity of such a DNA vaccine may be determined by standard pharmaceutical procedures in cell culture or in laboratory animals, for example, by ED50 (therapeutically effective dose in 50% of the population) or LD50 (lethal dose for 50% of the population) assay. Likewise, the effective dosage of the inducer of anti-Aβ antibody is an amount of the inducer required to induce detectable levels of anti-Aβ antibody in a biological sample (including blood, cell, tissue, etc.) collected from a patient. Detection of anti-Aβ antibody may be accomplished by immunological procedures such as ELISA, immunostaining and the like. Those skilled in the art would be able to determine an appropriate dosage for the DNA vaccine or the inducer of anti-Aβ antibody.

The route of administration may be selected as appropriate and examples include, but are not limited to, percutaneous, intranasal, transbronchial, intramuscular, intraperitoneal, intravenous and subcutaneous routes. Particularly preferred are intramuscular administration, subcutaneous administration and so on. Inoculation may be made at a single site or at multiple sites.

The dose ratio between therapeutic and toxic effects is a therapeutic index and can be expressed as ED50/LD50. In humans, the single dosage of the vaccine of the present invention or the inducer of anti-Aβ antibody according to the present invention is about 1 μg to 1000 μg, preferably about 10 to 500 μg, more preferably about 50 to 250 μg. The frequency of administration may be once or more as long as side effects are within a clinically acceptable range.

In the previous development of vaccines for Alzheimer's disease, studies have been conducted focusing on anti-Aβ antibody and Th2 activity. Thus, it is desirable to measure, in advance, the antibody titer or cellular immune activity as a vaccine.

For example, the cellular immune activity may be evaluated by isolating and culturing lymphocytes from the body and measuring their ³H-thymidine uptake.

Likewise, the Th2 activity may be evaluated by isolating plasma from peripheral blood and measuring its antibody titer by ELISA.

Once the DNA vaccine of the present invention is administered to a target animal, immune responses against Aβ are induced. Namely, since the above amino acid sequence of Aβ1-43, Aβ1-20, Aβ1-40 or Aβ1-42 contains an epitope, antibody production is induced when the vaccine of the present invention is administered.

Immune responses against Aβ may be detected by measuring the level of anti-Aβ antibody produced. The level of the antibody produced may be measured by standard immunological procedures such as ELISA (enzyme-linked immunosorbent assay). Likewise, the therapeutic effect of the vaccine may be confirmed, for example, as a reduction in the amount of Aβ in brain tissues or as a reduction in Aβ deposition (senile plaques). The amount of Aβ in brain tissues or the state of Aβ deposition may be observed by immunohistochemical or other techniques.

The present invention will be further described in more detail by way of the following illustrative examples, which are not intended to limit the scope of the invention.

### Example 1

1. Construction of Plasmid Vector pTarget/Ig-Leader-Aβ1-42-IgFc

### (1) Amplification and Cloning of IgL, Aβ and Fc Genes

To clone the sequences of immunoglobulin κ leader (hereinafter referred to as IgL) and immunoglobulin Fc (hereinafter referred to as Fc or IgFc), human peripheral blood mRNA was used as a material to synthesize cDNAs with ReverTra Ace-α-(TOYOBO, Tokyo, Japan). Primers containing the 5′- or 3′-terminal end of each sequence and having an appropriate restriction enzyme site (IgL: BamH I or Xho I; Fc: Kpn I or Not I) were designed and used to amplify human IgL and Fc sequences with KOD-plus-(Toyobo, Tokyo, Japan). Although the original sequence of human Fc contains three codons each encoding a cysteine residue near the 5′-terminal end, these codons were each modified to encode a serine residue (TGT→TCT or TGC→TCC) during primer design so as to avoid S—S linking, and the primers thus designed were used to obtain amplification products.

The sequence of amyloid β1-42 (hereinafter referred to as Aβ) was prepared by oligonucleotide synthesis, provided that two oligonucleotides were first synthesized, which contained the 5′- or 3′-terminal end of the Aβ sequence and were partially complementary to each other (24 bp in the middle of the Aβ sequence), because a sequence covering the full length (126 bp) was difficult to synthesize. An appropriate restriction enzyme site (Xho I, Kpn I) was added to each end. After these oligonucleotides were annealed, the entire duplex was prepared by polymerase reaction.

Each product obtained above was inserted into a cloning vector (IgL and Fc: Zero Blunt TOPO PCR Kit for Sequencing, Invitrogen, Tokyo, Japan; Ap: AdvanTAge PCR Cloning Kit, TAKARA, Tokyo, Japan), followed by sequencing to select a clone having the desired sequence.

(2) Insertion of IgL, Aβ and Fc Sequences into Expression Vector

DNAs of the IgL, Aβ and Fc sequences were excised from the cloning vectors by cleavage at the restriction enzyme sites previously added to each sequence, and then purified from an agarose gel (MinElute Gel Extraction Kit, Qiagen, Tokyo, Japan). Without using the TA cloning site, the pTarget vector was cleaved at two restriction enzyme sites Xho I and Kpn I located upstream and downstream of the TA cloning site to give the same overhangs as those of the Aβ sequence, and then purified from an agarose gel (MinElute Gel Extraction Kit).

First, the pTarget vector and the Aβ sequence were ligated together using a Ligation high kit (Toyobo, Tokyo, Japan) to construct pTarget/Aβ. Subsequently, the region immediately upstream of the Aβ sequence was treated with restriction enzymes BamH I and Xho I for integration of the IgL sequence to thereby construct pTarget/IgL-Aβ. Further, the Kpn I and Not I sites immediately downstream of the Aβ sequence were cleaved for ligation of the Fc sequence to complete pTarget/IgL-Aβ-Fc.

2. Preparation of pTarget/Ig Leader-Amyloid β 1-42-IL-4 (IgL-Aβ-IL-4) Vaccine

### (1) Amplification of IL-4 Sequence

Ig leader-Amyloid β1-42-IL-4 (IgL-Aβ-IL-4) (FIG. 1) was prepared using pTarget/Ig leader-Amyloid β 1-42-IgFc (IgL-Aβ-Fc) described above. First, human peripheral blood mRNA and mouse spleen mRNA were used to synthesize cDNAs with ReverTra Ace-α-(TOYOBO, Tokyo, Japan). Primers containing the 5′- or 3′-terminal end of human or mouse IL-4 mRNA (coding region) and having an appropriate restriction enzyme site (Kpn I or Not I) were designed and used to amplify the full-length human and mouse IL-4 sequences with KOD-plus-(Toyobo, Tokyo, Japan). PCR products were each purified from an agarose gel (MinElute Gel Extraction Kit, Qiagen, Tokyo, Japan) and inserted into a cloning vector (Zero Blunt TOPO PCR Kit for Sequencing, Invitrogen, Tokyo, Japan), followed by sequencing to select a clone having the desired sequence. For human IL-4, the sequences of both isotypes 1 (hIL-4) and 2 (hIL-4δ2), which were splice variants, were amplified. However, the latter is a receptor antagonist of IL-4 (Alms W J et al, Mol Immunol 1996, 33:361-370; Fletcher H A et al, Immunology 2004, 112:669-673; Plante S. et al, J Allergy Clin Immunol 2006, 117:1321-1327), and hence isotype 1 was selected.

(2) Recombination to Replace Fc Sequence with IL-4 Sequence

The IL-4 sequence was excised from the cloning vector by cleavage at the previously added restriction enzyme sites (Kpn I, Not I), while the Fc sequence was excised from pTarget/IgL-Aβ-Fc with the same restriction enzyme pair. The hIL-4 or mIL-4 sequence and pTarget/IgL-Aβ were each purified from an agarose gel (MinElute Gel Extraction Kit) and then ligated together overnight at 16° C. using a Ligation high kit (Toyobo, Tokyo, Japan), followed by transformation into TOP10F′ (Invitrogen, Tokyo, Japan) and blue-white selection to select a clone carrying the desired sequence (i.e., an insert of the correct size) by restriction enzyme cleavage. The nucleotide sequence of the insert in pTarget/IgL-Aβ1-42-hIL-4 thus obtained is shown in SEQ ID NO: 21, while the nucleotide sequence of the insert in pTarget/IgL-Aβ1-42-mIL-4 thus obtained is shown in SEQ ID NO: 22.

3. Preparation of pIRES2/Ig Leader-Amyloid β1-42-M-CSF (IgL-Aβ-M-CSF) Vaccine

Since M-CSF is a protein that exerts its functions upon homodimer formation, Aβ1-42 and M-CSF were expressed as separate proteins with an IRES system.

### (1) Amplification of M-CSF Sequence and Ig Leader (IgL) Sequence

Mouse spleen mRNA was used to synthesize cDNA with ReverTra Ace-α-(TOYOBO, Tokyo, Japan). Primers containing the 5′- or 3′-terminal end of mouse M-CSF (moM-CSF) mRNA (coding region) and having any appropriate restriction enzyme site (BamH I or Not I) were designed and used to amplify the full-length mouse M-CSF sequence with KOD-plus-(Toyobo, Tokyo, Japan). The Ig leader (IgL) sequence was amplified with primers having necessary restriction enzyme sites (Nhe I, Xho I) by using pTarget/Ig leader-Amyloid β1-42-IgFc (pTarget/IgL-Aβ-Fc) as a template. The PCR products were each purified from an agarose gel (MinElute Gel Extraction Kit, Qiagen, Tokyo, Japan) and inserted into a cloning vector (Zero Blunt TOPO PCR Kit for Sequencing, Invitrogen, Tokyo, Japan), followed by sequencing to select a clone having the desired sequence.

(2) Recombination to Replace APL Sequence with IgL Sequence

Based on the previously prepared vector pIRES2-EGFP/Alkaline phosphatase leader-Amyloid β 1-42 (pIRES2-EGFP/APL-Aβ) (pIRES2-EGFP vector: Takara-Chlontech, Tokyo, Japan), pIRES2-EGFP/Ig leader-Amyloid β1-42 (pIRES2-EGFP/IgL-Aβ) was first prepared. The APL sequence was excised by cleavage at the restriction enzyme sites (Nhe I, Xho I) added to the both ends of this sequence, while the IgL sequence was excised from the cloning vector with the same restriction enzyme pair. pIRES2-EGFP/Aβ and the IgL sequence were each purified from an agarose gel (MinElute Gel Extraction Kit) and then ligated together overnight at 16° C. using a Ligation high kit (Toyobo, Tokyo, Japan), followed by transformation into TOP10 (Invitrogen, Tokyo, Japan) to select a clone carrying the desired sequence (i.e., an insert of the correct size) by restriction enzyme cleavage.

(3) Recombination to Replace EGFP Sequence with M-CSF Sequence

Next, the EGFP sequence was excised from pIRES2-EGFP/IgL-Aβ by cleavage at the restriction enzyme sites (BstX I, Not I), while the M-CSF sequence was excised from the cloning vector by restriction enzyme cleavage (BamH I, Not I). After pIRES2/IgL-Aβ and M-CSF were each purified from an agarose gel, the overhangs of both constructs were blunt-ended with KOD DNA polymerase (Toyobo, Tokyo, Japan). pIRES2/IgL-Aβ was treated with alkaline phosphatase (E. coli alkaline phosphatase, Toyobo, Tokyo, Japan) and then cleaned up (QIAquick PCR Purification Kit, Qiagen, Tokyo, Japan). These two constructs were ligated together using a Ligation high kit (Toyobo, Tokyo, Japan) to complete pIRES2/IgL-Aβ-moM-CSF.

The nucleotide sequence of the insert in pIRES2/IgL-Aβ-moM-CSF thus obtained is shown in SEQ ID NO: 23.

4. Preparation of pVAX1/IgL-Aβ1-42-huIgFc-huIL-4 and pVAX1/IgL-Aβ1-42-mIgFc-mIL-4 Vaccines
(1) Preparation of huIgFc-huIL-4 and mIgFc-mIL-4 Constructs

To amplify huIgFc, pTarget/IgL-Aβ1-42-huIgFc was used as a template for PCR with primers which were designed to have appropriate restriction enzyme sites (Kpn I and Sal I) and to remove the stop codon.

To amplify mIgFc, cDNA derived from mRNA extracted from mouse spleens was used as a template for PCR with primers which were designed to have restriction enzyme sites (Kpn I and Sal I) and to remove the stop codon.

It should be noted that although the original IgFc sequence contains codons each encoding a cysteine residue (Cys), the primers were designed such that seven Cys residues were each replaced with Ser so as to avoid S—S linking.

To amplify huIL-4 and mIL-4, pTarget/IgL-Aβ1-42-huIL-4 and pTarget/IgL-Aβ1-42-mM-4 were used as templates, respectively, for PCR with primers which were designed to remove their respective leader sequences (signal peptides) and to have a linker sequence (TCTTCTGGTGGTGGTGGT; SEQ ID NO: 24) on the 5′-side. In this case, each construct was amplified such that restriction enzyme sites (Sal I and Not I) were newly added and the stop codon found in the template was maintained.

The amplified gene fragments of huIL-4 and mIL-4 were cleaved at the previously added restriction enzyme sites. Pairs between huIgFc and huIL-4 and between mIgFc and mIL-4 were each ligated using a Ligation high kit (Toyobo, Tokyo, Japan) and cloned (pCR4-Blunt TOPO; Invitrogen, Tokyo, Japan), followed by sequencing to select a clone having the correct sequence of huIgFc-huIL-4 or mIgFc-mIL-4.

(2) Construction of pVAX1/IgL-Aβ1-42-huIgFc-huIL-4 and pVAX1/IgL-Aβ1-42-mIgFc-mIL-4

The huIgFc-huIL-4 or mIgFc-mIL-4 construct was excised by cleavage at the terminal restriction enzyme sites (Kpn I and Not I) and used for recombination with the huIL-4 moiety of pTarget/IgL-Aβ1-42-huIL-4, which had been excised by cleavage at the same sites. As a result, pTarget/IgL-Aβ1-42-huIgFc-huIL-4 and pTarget/IgL-Aβ1-42-mIgFc-mIL-4 were obtained. Further, IgL-Aβ1-42-huIgFc-huIL-4 and IgL-Aβ1-42-mIgFc-mIL-4 were each excised from the pTarget vector by cleavage at the terminal restriction enzyme sites (BamH I and Not I) and then ligated using a Ligation high kit (Toyobo, Tokyo, Japan) to a pVAX1 vector (Invitrogen, Tokyo, Japan) which had been treated with the same restriction enzyme pair, thereby completing pVAX1/IgL-Aβ1-42-huIgFc-huIL-4 and pVAX1/IgL-Aβ1-42-mIgFc-mIL-4. The nucleotide sequences of the inserts in pVAX1/IgL-Aβ1-42-huIgFc-huIL-4 and pVAX1/IgL-Aβ1-42-mIgFc-mIL-4 thus obtained are shown in SEQ ID NOs: 25 and 26, respectively.

5. Preparation of pVAX1/IgL-(Aβ1-42)x4-huIgFc-huIL-4 and pVAX1/IgL-(Aβ1-42)x4-moIgFc-mIL-4 vaccines

A construct carrying 4 repeats of the Aβ1-42 gene ((Aβ1-42)x4) was prepared by linking 4 units of Aβ1-42 via 3 linker sequences (GGTGGCGGTGGCTCG: SEQ ID NO: 27). First, two constructs, i.e., Aβ1-42+linker sequence+Aβ1-6 and Aβ37-42+linker sequence+Aβ1-42 were prepared by PCR amplification. Next, both constructs were mixed together and used as a template for PCR amplification with a sense primer designed to have a restriction enzyme site Xho I on the 5′-side of Aβ1-42 and an antisense primer designed to have a restriction enzyme site Kpn I on the 3′-side of Aβ1-42. The amplification products were electrophoresed on an agarose gel. Among bands in a ladder pattern, a band of about 560 bp corresponding to the molecular weight of (Aβ1-42)x4 was excised and purified.

The (Aβ1-42)x4 construct thus prepared was cloned and sequenced, and then excised by cleavage at the previously added restriction enzyme sites (Xho I and Kpn I) and used for recombination with the Aβ1-42 moiety of pTarget/IgL-Aβ1-42-huIgFc-huIL-4 or pTarget/IgL-Aβ1-42-mIgFc-mIL-4, which had been excised by cleavage at the same sites. As a result, pTarget/IgL-(Aβ1-42)x4-huIgFc-huIL-4 and pTarget/IgL-(Aβ1-42)x4-mIgFc-mIL-4 were obtained. Further, IgL-(Aβ1-42)x4-huIgFc-huIL-4 and IgL-(Aβ1-42)x4-mIgFc-mIL-4 were each excised from the pTarget vector by cleavage at the terminal restriction enzyme sites (BamH I and Not I) and then ligated using a Ligation high kit (Toyobo, Tokyo, Japan) to a pVAX1 vector (Invitrogen, Tokyo, Japan) which had been treated with the same restriction enzyme pair, thereby completing pVAX1/IgL-(Aβ1-42)x4-huIgFc-huIL-4 and pVAX1/IgL-(Aβ1-42)x4-mIgFc-mIL-4. The nucleotide sequences of the inserts in pVAX1/IgL-(Aβ1-42)x4-huIgFc-huIL-4 and pVAX1/IgL-(Aβ1-42)x4-mIgFc-mIL-4 thus obtained are shown in SEQ ID NOs: 28 and 29, respectively.

6. Preparation of pVAX1/IgL-(Aβ1-42)x4-huIgFc and pVAX1/IgL-(Aβ1-42)x4-mIgFc Vaccines

To amplify huIgFc and mIgFc each carrying a stop codon, pTarget/IgL-Aβ1-42-huIgFc-huIL-4 and pTarget/IgL-Aβ1-42-mIgFc-mIL-4 were used as templates, respectively, for PCR with primers which were designed to have appropriate restriction enzyme sites (Kpn I and Not I) and a stop codon. The amplification products were each cloned and sequenced, and then excised by cleavage at the restriction enzyme sites (Kpn I and Not I) and used for recombination with the huIgFc-huIL-4 moiety of pTarget/IgL-(Aβ1-42)x4-huIgFc-huIL-4 or with the mIgFc-mIL-4 moiety of pTarget/IgL-(Aβ1-42)x4-mIgFc-mIL-4, which had been excised by cleavage at the same sites. As a result, pTarget/IgL-(Aβ1-42)x4-huIgFc and pTarget/IgL-(Aβ1-42)x4-mIgFc were obtained. Further, IgL-(Aβ1-42)x4-huIgFc and IgL-(Aβ1-42)x4-mIgFc were each excised from the pTarget vector by cleavage at the terminal restriction enzyme sites (BamH I and Not I) and then ligated using a Ligation high kit (Toyobo, Tokyo, Japan) to a pVAX1 vector (Invitrogen, Tokyo, Japan) which had been treated with the same restriction enzyme pair, thereby completing pVAX1/IgL-(Aβ1-42)x4-huIgFc and pVAX1/IgL-(Aβ1-42)x4-mIgFc. The nucleotide sequences of the inserts in pVAX1/IgL-(Aβ1-42)x4-huIgFc and pVAX1/IgL-(Aβ1-42)x4-mIgFc thus obtained are shown in SEQ ID NOs: 30 and 31, respectively.

### Example 2

### Vaccination Test

1. Vaccination test with pTarget/IgL-Aβ1-42-mIL-4 and pIRES2/IgL-Aβ1-42-moM-CSF

### (1) Materials and Methods

In the vaccination test, model mice of Alzheimer's disease were used. These model mice were obtained from the Jackson Laboratory, USA. The vaccines used were pTarget/IgL-Aβ1-42-mIL-4 (also referred to as “Aβ-IL4 vaccine”), which was prepared by integrating mouse IL-4 into the plasmid, and pIRES2/IgL-Aβ1-42-moM-CSF (also referred to as “M-CSF vaccine”), which was prepared by integrating mouse M-CSF into the plasmid.

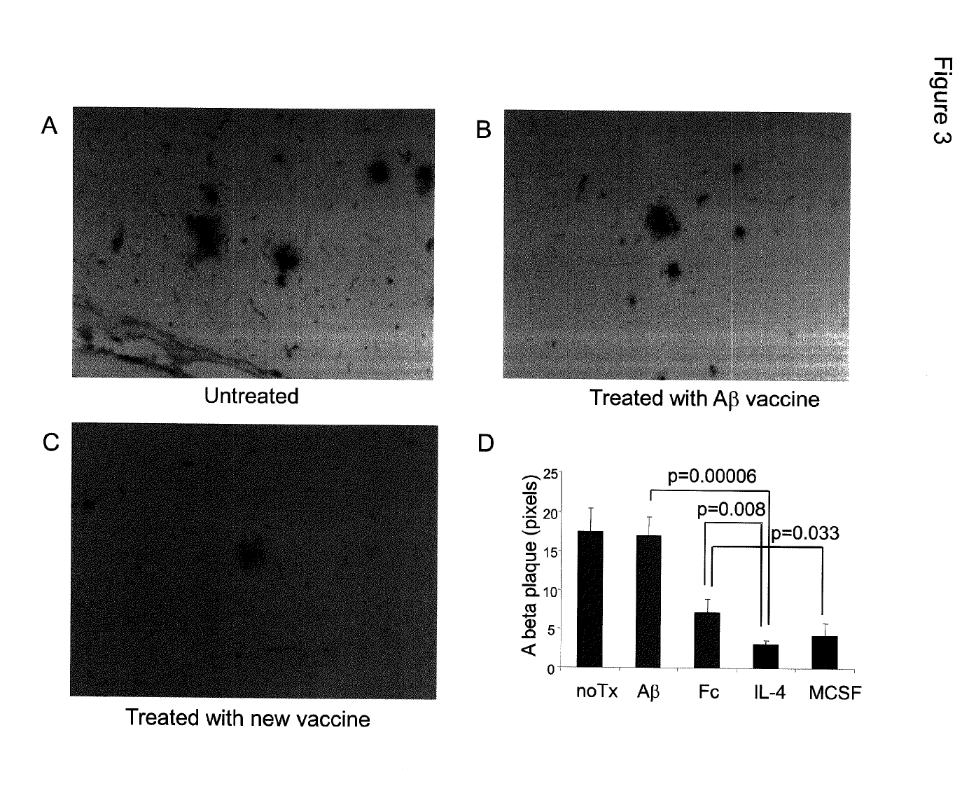

The model mice at 4 months of age were vaccinated (100 μg) once per two weeks by intramuscular injection, and the effect of eliminating deposited Aβ was observed at 10 months of age (FIG. 3). First, the mice were sacrificed under deep anesthesia, and their cerebrums were excised and fixed with 4% paraformaldehyde. The fixed brains were dehydrated, embedded in paraffin, and then sliced into thin sections. The sections were deparaffinized and then immunostained with anti-Aβ antibody (FIGS. 3A to 3C). Ten microscopic fields of the brain cortex and hippocampus were randomly photographed from each stained section, and quantified as the total area of Aβ deposition by using NIH image software (FIG. 3D).

The total amount and properties of antibody in the vaccinated animals were analyzed by ELISA and Aβ affinity assay, respectively. ELISA was performed in a standard manner, while the Aβ affinity assay was performed as follows. A test plasma was serially diluted and the resulting respective samples were contacted with cerebral sections of each model mouse to determine the presence or absence of sample binding in an immunohistological manner. The maximum dilution factor giving a positive result was defined as Aβ affinity activity of the sample.

### (2) Results

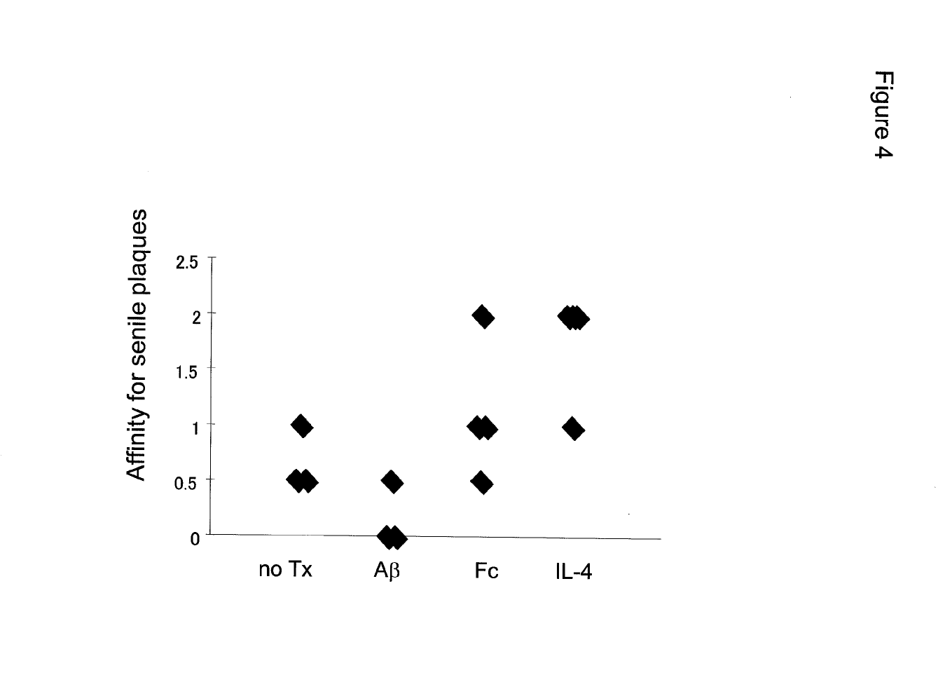

The results obtained are shown in FIGS. 3 and 4. In FIGS. 3D and 4, “noTx” represents the untreated group, “Aβ” represents the group receiving Aβ vaccination, and “Fc” represents the group receiving Aβ-Fc vaccination. Likewise, “IL-4” represents the group receiving Aβ-IL4 vaccination, and “MCSF” represents the group receiving M-CSF vaccination.

In FIG. 3, when compared to the untreated group (FIG. 3A), the group receiving existing vaccination (Aβ vaccine, FIG. 3B) shows an Aβ-eliminating effect, which is not so remarkable. In contrast, the new vaccine (Aβ-IL4 vaccine) was clearly found to have a stronger Aβ-eliminating effect (FIG. 3C). These results were quantified and analyzed, indicating that the Aβ-IL4 vaccine had a significantly strong Aβ-eliminating effect, when compared to the existing vaccines (Aβ vaccine, Aβ-Fc vaccine (IgL-Aβ-Fc)) (FIG. 3D). Moreover, the M-CSF vaccine was also found to have a remarkable Aβ-eliminating effect (FIG. 3D).

FIG. 4 indicated that the mice receiving Aβ-IL4 vaccination showed increased levels of antibody with high affinity for Aβ deposition (senile plaques), although there was no significant difference over the control group in their anti-Aβ-IgG levels (FIG. 4). The nature of this induced antibody would support the high Aβ-eliminating effect produced by the new Aβ-IL4 vaccine.

2. Vaccination Test with pVAX1/IgL-(Aβ1-42)x4-huIgFc-huIL-4 (YM3711) and Others

### (1) Materials and Methods

pVAX1/IgL-Aβ1-42-huIgFc-huIL-4 (FIG. 5), pVAX1/IgL-Aβ1-42-mIgFc-mIL-4 (FIG. 6), pVAX1/IgL-(Aβ1-42)x4-huIgFc-huIL-4 (FIG. 7), pVAX1/IgL-(Aβ1-42)x4-mIgFc-mIL-4 (FIG. 8), pVAX1/IgL-(Aβ1-42)x4-huIgFc (FIG. 9) and pVAX1/IgL-(Aβ1-42)x4-mIgFc (FIG. 10) were newly constructed and used in the vaccination test.

In the vaccination test, model mice of Alzheimer's disease were used. These model mice were obtained from the Jackson Laboratory, USA.

The vaccines used for mice were pVAX1/IgL-Aβ1-42-mIgFc-mIL-4, pVAX1/IgL-(Aβ1-42)x4-mIgFc-mIL-4 and pVAX1/IgL-(Aβ1-42)x4-mIgFc. The vaccines used for rabbits were pVAX1/IgL-Aβ1-42-huIgFc-huIL-4, pVAX1/IgL-(Aβ1-42)x4-huIgFc-huIL-4 and pVAX1/IgL-(Aβ1-42)x4-mIgFc.

First, the above vaccines were each introduced into cultured cells. The cells were incubated for 3 days and then analyzed by sandwich ELISA to determine the amounts of Aβ present within the cells and in the culture supernatant. The cultured cells used were HEK392 cells.

Next, the model mice at 10 months of age were vaccinated (100 μg) once a week by intramuscular injection, and blood was sampled over time from their orbita. Plasma was separated and analyzed by ELISA to determine the titer of anti-Aβ antibody. In the same manner, rabbits were also vaccinated and measured for antibody titer in their plasma.

The model mice were sacrificed at 12 months of age to study the effect of vaccination on elimination of Aβ deposited in the brain. First, cervical dislocation was performed under deep anesthesia, and cerebrums were excised and fixed with 4% paraformaldehyde. The fixed brains were dehydrated, embedded in paraffin, and then sliced into thin sections. The sections were deparaffinized and then immunostained with anti-Aβ antibody. Ten microscopic fields of the brain cortex were randomly photographed from each stained section, and quantified as the total area of Aβ deposition by using NIH image software.

### (2) Results

The representative results are shown in FIGS. 11 to 13.

In FIG. 11, “IgL-Aβ-IL4” represents pVAX1/IgL-Aβ1-42-mIL-4, and “IgL-Aβ-Fc-IL4” represents pVAX1/IgL-Aβ1-42-mIgFc-mIL-4. Likewise, “cell pellet” represents the cell pellet fraction, and “sup.” represents the culture supernatant fraction.

Upon introduction into cultured cells, the vaccine carrying immunoglobulin Fc sequence (“IgL-Aβ-Fc-IL4”) showed a several-fold or more increase in Aβ production within the cultured cells (“cell pellet”), when compared to the Fc sequence-free vaccine (“IgL-Aβ-IL4”). Moreover, the Fc sequence-free vaccine caused little Aβ secretion into the culture supernatant, whereas the Fc sequence-carrying vaccine was found to cause remarkable secretion (“sup.”).

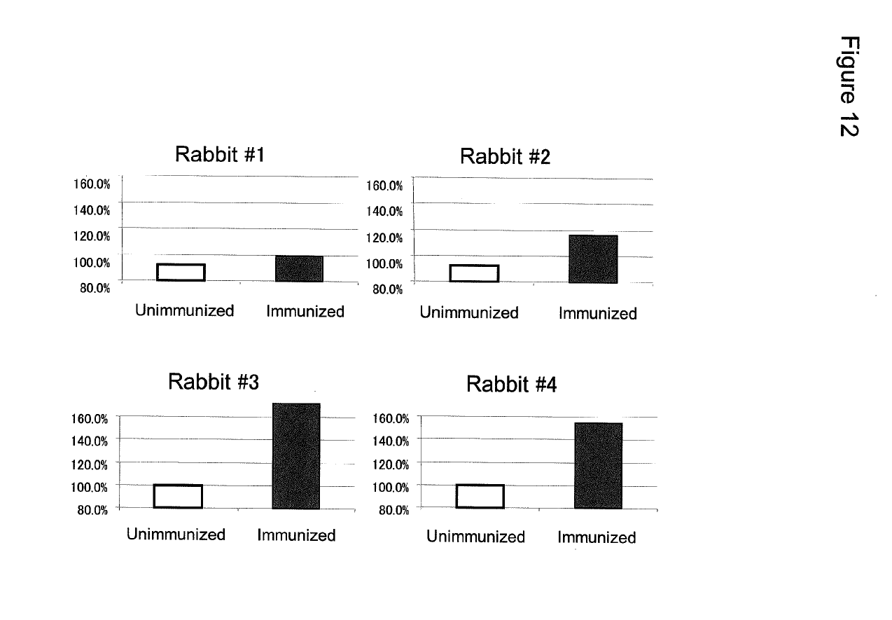

When rabbits were administered with pVAX1/IgL-Aβ1-42-huIgFc-huIL-4 (hereinafter also referred to as “Aβ(1-42)x1 vaccine”) and pVAX1/IgL-(Aβ1-42)x4-huIgFc-huIL-4 (hereinafter also referred to as “YM3711”), not only the Aβ(1-42)x1 vaccine carrying no repeat of Aβ1-42, but also YM3711 carrying repeats of Aβ1-42 caused significant induction of anti-Aβ antibody (FIG. 12). Moreover, the rabbits administered with YM3711 carrying repeats of Aβ1-42 (“Rabbits #3 and #4” in FIG. 12) showed higher antibody titers than the rabbits administered with the Aβ(1-42)x1 vaccine carrying no repeat of Aβ1-42 (“Rabbits #1 and #2” in FIG. 12) (FIG. 12). This result indicates that the vector of the present invention is useful as an inducer of anti-Aβ antibody.

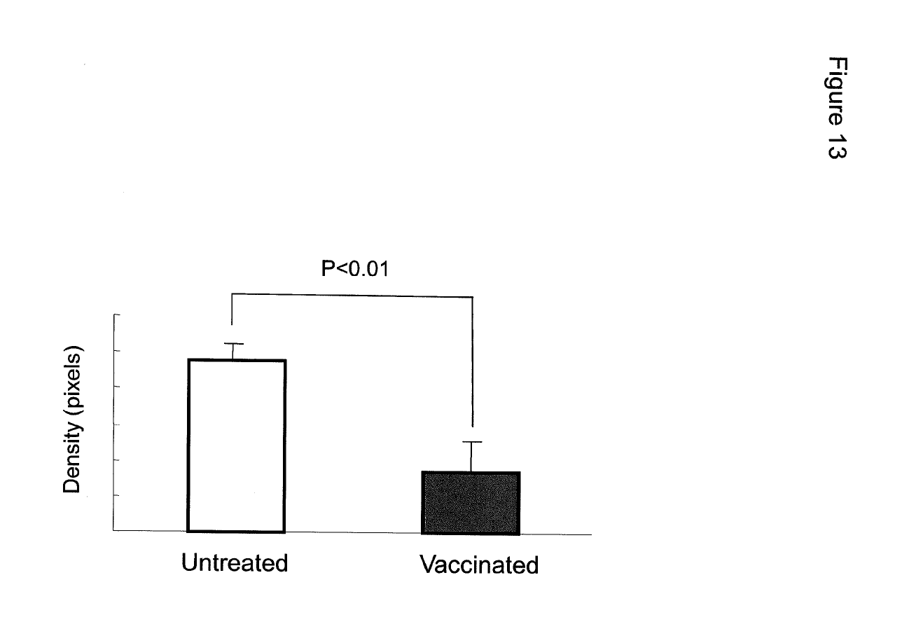

In the quantification of brain Aβ deposition, the mice administered with pVAX1/IgL-(Aβ1-42)x4-mIgFc-mIL-4 clearly showed a remarkable Aβ-eliminating effect, in comparison with the untreated group (FIG. 13).

Until now, some attempts have been made to treat Alzheimer model mice with a vaccine prepared by introducing a gene for Aβ1-42, IL-4 or tissue plasminogen activator into a plasmid vector. However, such a DNA vaccine alone was not sufficient to induce a decrease in brain Aβ levels. This would be because the structure of these vaccines has a defect in the mechanism for elevating anti-Aβ antibody levels. As shown above, the vaccines prepared by the inventors of the present invention were found to be effective by themselves and to produce a higher Aβ-eliminating effect than other existing vaccines. In particular, the human and mouse types of pVAX1/IgL-(Aβ1-42)x4-IgFc-IL-4 vaccines were shown to have an extremely high Aβ-eliminating effect.

3. Study on pVAX1/IgL-(Aβ1-42)x4-huIgFc-huIL-4 (YM3711) for its Ability to Induce Various Antibodies

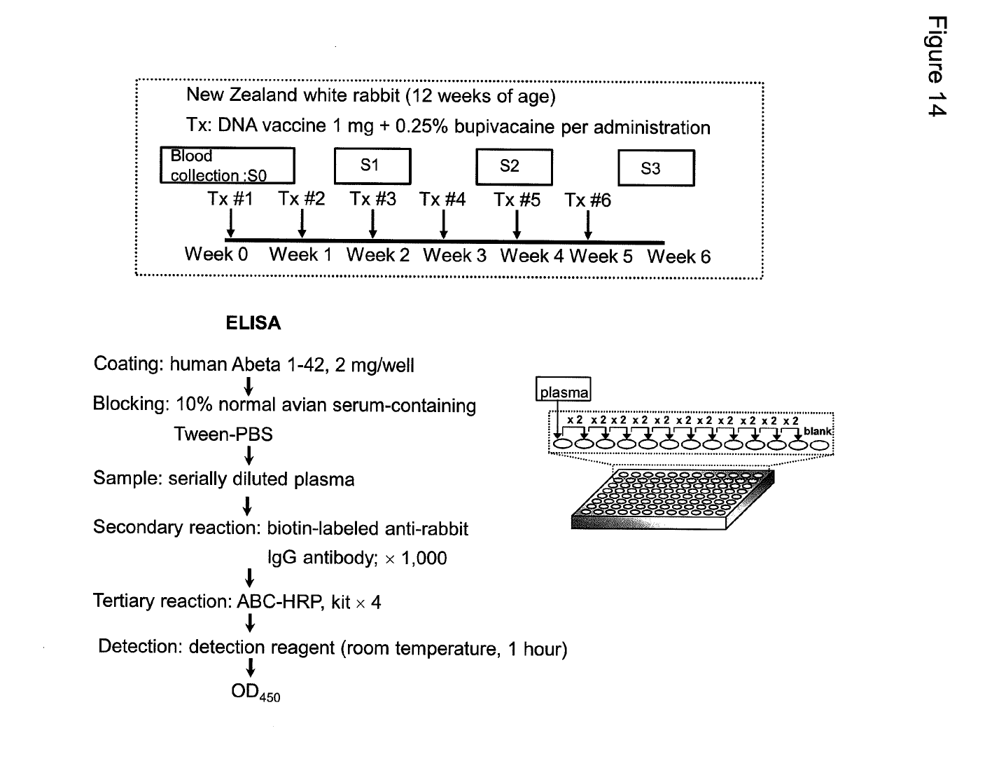

The inventors of the present invention studied pVAX1/IgL-(Aβ1-42)x4-huIgFc-huIL-4 (YM3711) for its ability to induce various antibodies in the same manner as used in the vaccination test in 2 above. More specifically, rabbits were injected six times (once a week for 6 weeks) with 1 mg of YM3711 by the intramuscular route, and blood was sampled over time. Plasma was separated and analyzed by ELISA to determine the titer of anti-Aβ antibody in the plasma, which was then compared with the unimmunized plasma. The same test was also repeated using an existing vaccine (pVAX1/IgL-Aβ1-42-huIgFc), and the degree of antibody titer in this case was compared with that of YM3711. FIG. 14 shows the schedule of DNA vaccination and procedures for antibody titer assay in rabbits.

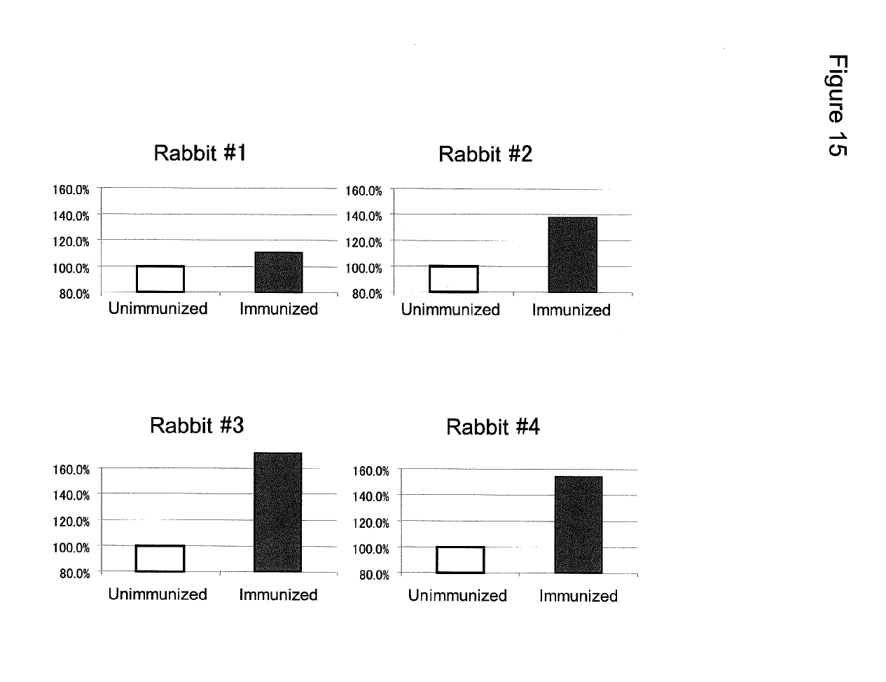

As a result, the rabbits administered with YM3711 (“Rabbits #3 and #4” in FIG. 15) were found to show a higher increase in the antibody titer of anti-Aβ1-42 antibody than the rabbits administered with the existing vaccine (“Rabbits #1 and #2” in FIG. 15) (FIG. 15). Table 1 below shows the % increase in antibody titer after immunization with YM3711.

TABLE 1
Increase (%) in antibody titer after immunization

| | |
|---|---|
| Anti-Aβ1-42 antibody | (%) |

| | | |
|---|---|---|
| | Rabbit #1 | Rabbit #2 |
| Existing vaccine | 12 | 39 |
| | Rabbit #3 | Rabbit #4 |
| YM3711 | 68 | 58 |

As shown in Table 1, the rabbits administered with the existing vaccine (“Rabbits #1 and #2” in Table 1) showed an increase of 12% to 39% when compared to before immunization. In contrast, the rabbits administered with YM3711 (“Rabbits #3 and #4” in Table 1) showed an increase as high as 58% to 68%. This indicates that YM3711 causes remarkable induction of anti-Aβ1-42 antibody in rabbits, in comparison with the existing vaccine.

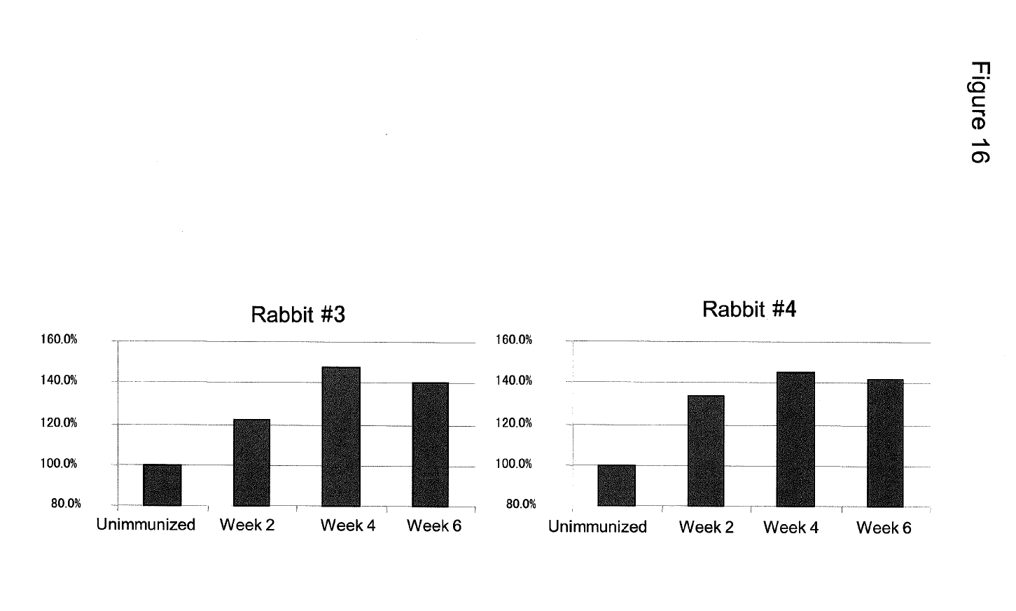

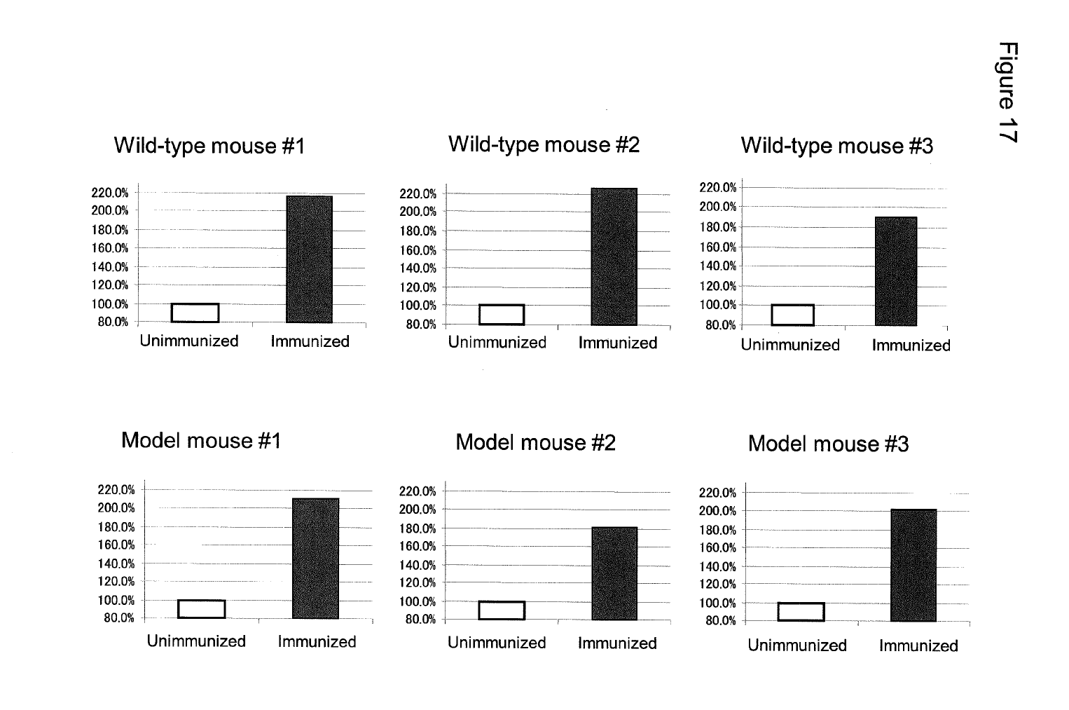

The antibody titer of anti-Aβ1-42 antibody started to increase at 2 weeks after immunization and was then maintained at a high level (FIG. 16). Moreover, not only the rabbits, but also wild-type or model mice showed an increase in antibody titer upon DNA vaccination (FIG. 17). Table 2 below shows the % increase in antibody titer after wild-type and model mice were each immunized with YM3711.

TABLE 2
Anti-Aβ antibody-inducing effect of YM3711 in mice

| | (%) | | |
|---|---|---|---|
| | #1 | #2 | #3 |
| Wild-type mouse | 119 | 124 | 91 |
| Model mouse | 111 | 81 | 100 |

As shown in Table 2, YM3711 was found to cause an increase of 91% to 124% in the wild-type mice and 81% to 111% in the model mice. This indicates that YM3711 also causes remarkable induction of anti-Aβ1-42 antibody in both wild-type and model mice.

The inventors of the present invention further conducted the same test in monkeys, which are most important in drug development. More specifically, male cynomolgus monkeys at 3.5 to 4.5 years of age (3.0 to 4.0 kg) were injected with 0.5 mg/kg of YM3711 once a week by the intramuscular route, and blood was collected before immunization and after 2 and 4 weeks. Plasma fractions of these blood samples were used in ELISA using the Aβ1-42 peptide as an antigen to determine their anti-Aβ antibody titer. In this test, the antibody titer before immunization was set to 100%, and % elevation (% increase) of antibody titer was calculated for each animal.

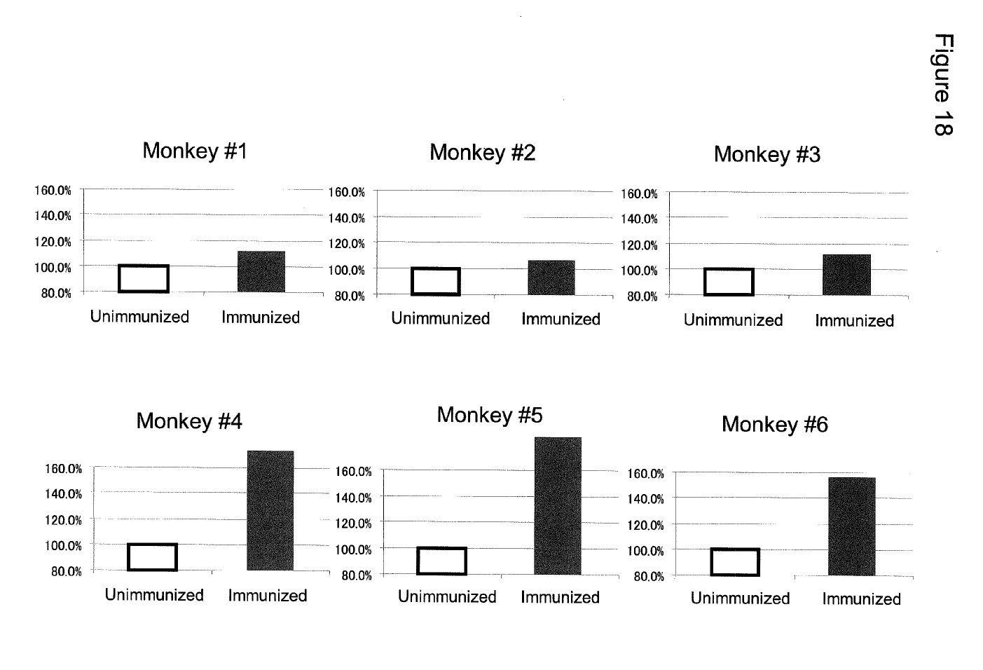

As a result, the monkeys administered with YM3711 (“Monkeys #4 to #6” in FIG. 18) were found to show a remarkably higher increase in the antibody titer of anti-Aβ1-42 antibody than the monkeys administered with the existing vaccine (pVAX1/IgL-Aβ1-42-huIgFc) (“Monkeys #1 to #3” in FIG. 18) (FIG. 18). Table 3 below shows the % increase in antibody titer after immunization with YM3711.

TABLE 3
Increase (%) in antibody titer after immunization

| Anti-Aβ1-42 antibody | (%) | | |
|---|---|---|---|
| | Monkey #1 | Monkey #2 | Monkey #3 |
| Existing vaccine | 12 | 8 | 12 |
| | Monkey #4 | Monkey #5 | Monkey #6 |
| YM3711 | 75 | 81 | 58 |

As shown in Table 3, the monkeys administered with the existing vaccine (“Monkeys #1 to #3” in Table 3) showed an increase of 8% to 12% when compared to before immunization. In contrast, the monkeys administered with YM3711 (“Monkeys #4 to #6” in Table 3) showed an increase as high as 58% to 81%. This indicates that YM3711 causes remarkable induction of anti-Aβ1-42 antibody in monkeys, in comparison with the existing vaccine.

Moreover, in comparison with the test in rabbits (FIG. 15 and Table 1), the monkeys showed a remarkable difference between the increase in antibody titer after immunization with the existing vaccine and the increase in antibody titer after immunization with YM3711. This indicates that YM3711 is a vaccine or an inducer of anti-Aβ1-42 antibody, which is more compatible than the existing vaccine with animals closer to humans.

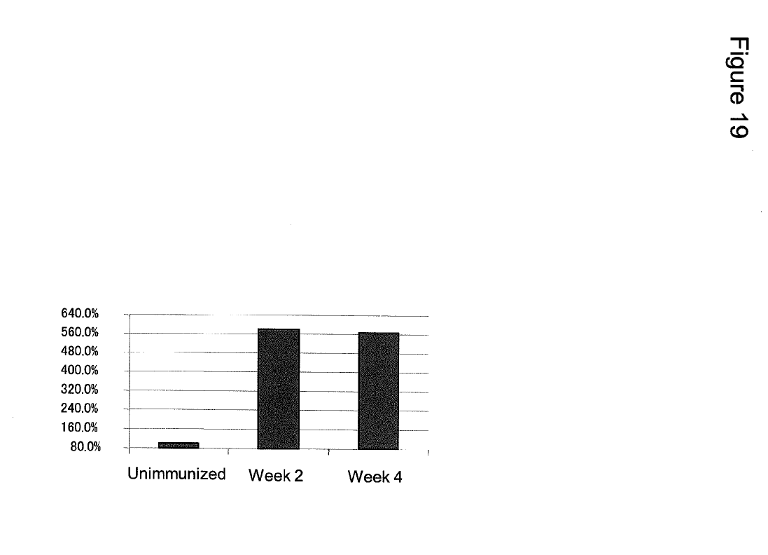

In the monkeys, the antibody titer of anti-Aβ1-42 antibody started to increase at 2 weeks after immunization and was then maintained at a high level, which indicated a sufficient increase in anti-Aβ antibody levels until 4 weeks after immunization (FIG. 19).

Moreover, to study the ability of YM3711 to induce various antibodies, the same procedure as used above in antibody titer assay for anti-Aβ1-42 antibody was repeated to determine the antibody titers of other antibodies, i.e., antibody against pEAβ3-42 having strong neurotoxicity, as well as antibodies against ABri and ADan having high amyloid aggregation propensity although their amino acid sequences are completely different from that of Aβ. Antibody titer assay was performed with rabbits and monkeys.

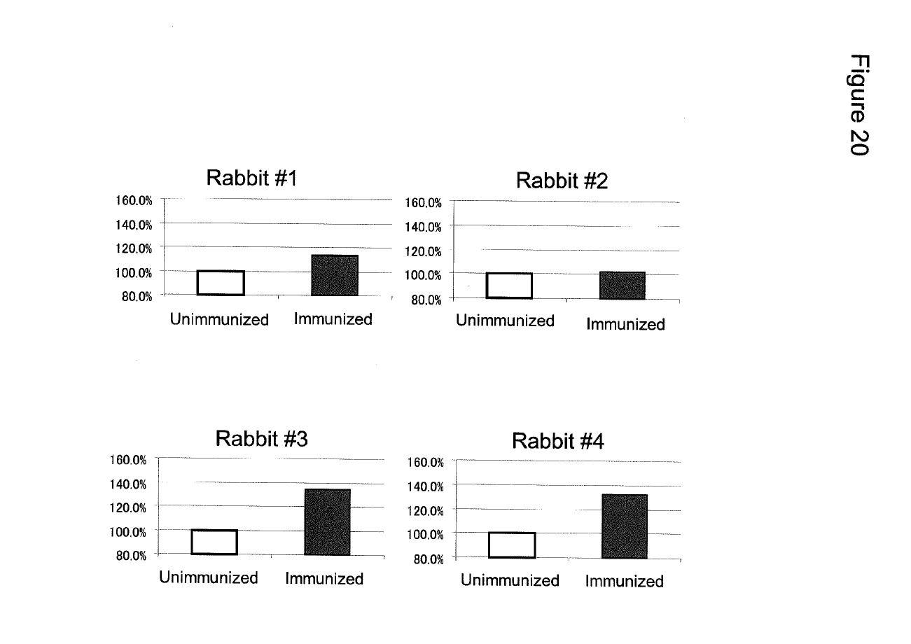

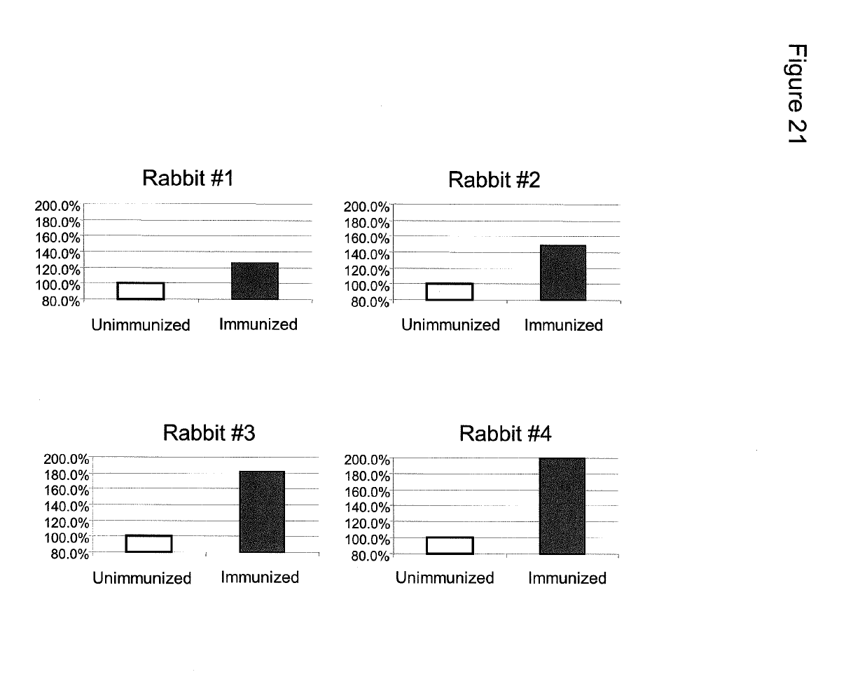

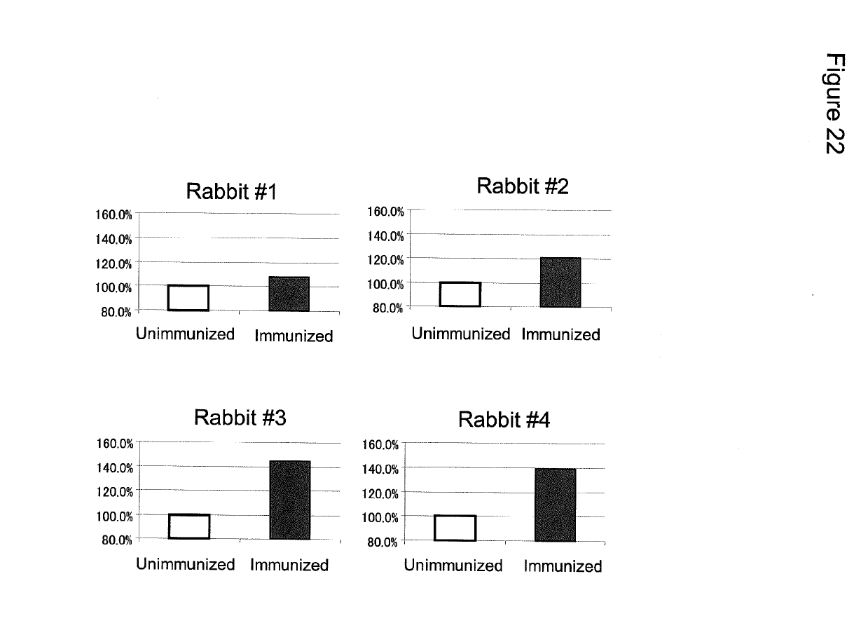

The results obtained with rabbits are shown in FIGS. 20 to 22. FIG. 20 shows the anti-pEAβ3-42 antibody-inducing effect produced by YM3711, FIG. 21 shows the anti-ABri antibody-inducing effect produced by YM3711, and FIG. 22 shows the anti-ADan antibody-inducing effect produced by YM3711. In FIGS. 20 to 22, “Rabbits #1 and #2” show the results in rabbits administered with the existing vaccine (pVAX1/IgL-Aβ1-42-huIgFc), while “Rabbits #3 and #4” show the results in rabbits administered with YM3711.

These results indicated that YM3711 induced anti-pEAβ33-42 antibody, anti-ABri antibody and anti-ADan antibody in the rabbits administered therewith. Table 4 below shows the % increase in antibody titer after immunization with YM3711.

TABLE 4
Increase (%) in antibody titer after immunization

(%)

| | | |
|---|---|---|
| Anti-pEAβ3-42 antibody | | |
| | Rabbit #1 | Rabbit #2 |
| Existing vaccine | 18 | 1 |
| | Rabbit #3 | Rabbit #4 |
| YM3711 | 38 | 37 |
| Anti-ABri antibody | | |
| | Rabbit #1 | Rabbit #2 |
| Existing vaccine | 26 | 50 |
| | Rabbit #3 | Rabbit #4 |
| YM3711 | 81 | 100 |
| Anti-ADan antibody | | |
| | Rabbit #1 | Rabbit #2 |
| Existing vaccine | 8 | 20 |
| | Rabbit #3 | Rabbit #4 |
| YM3711 | 43 | 39 |

As shown in Table 4, the rabbits administered with the existing vaccine (“Rabbits #1 and #2” in Table 4) showed an increase of 1% to 18% for anti-pEAβ3-42 antibody, 26% to 50% for anti-ABri antibody, and 8% to 20% for anti-ADan antibody, when compared to before immunization. In contrast, the rabbits administered with YM3711 (“Rabbits #3 and #4” in Table 4) showed an increase of 37% to 38% for anti-pEAβ3-42 antibody, 81% to 100% for anti-ABri antibody, and 39% to 43% for anti-ADan antibody. This indicates that YM3711 causes remarkable induction of anti-pEAβ3-42 antibody, anti-ABri antibody and anti-ADan antibody in rabbits, in comparison with the existing vaccine.

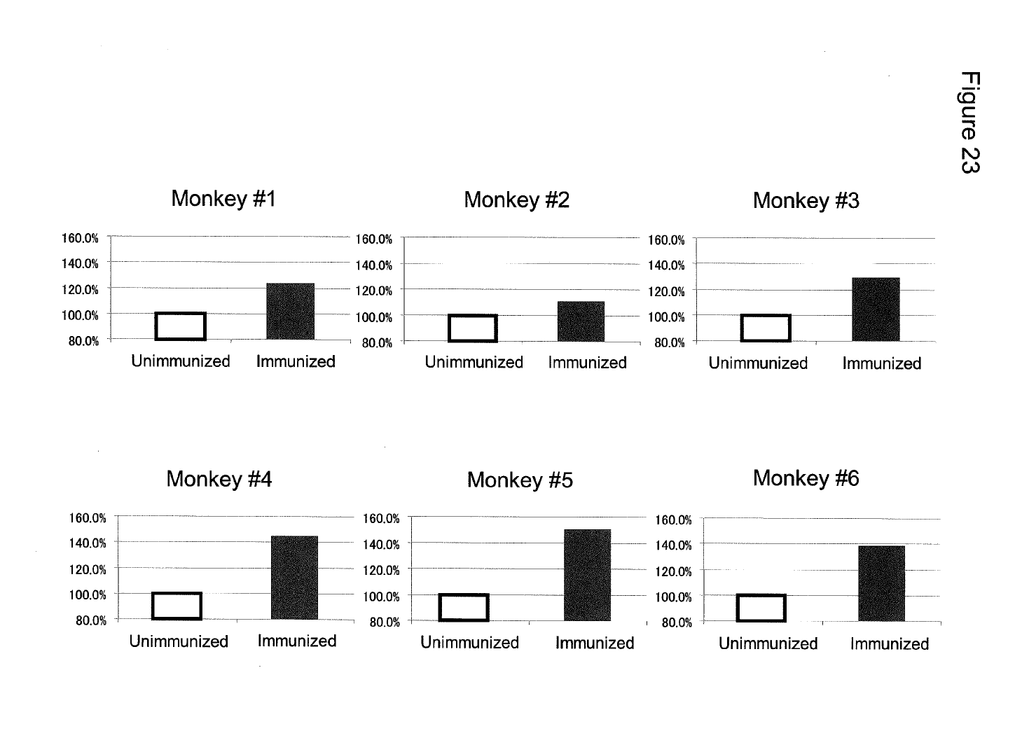

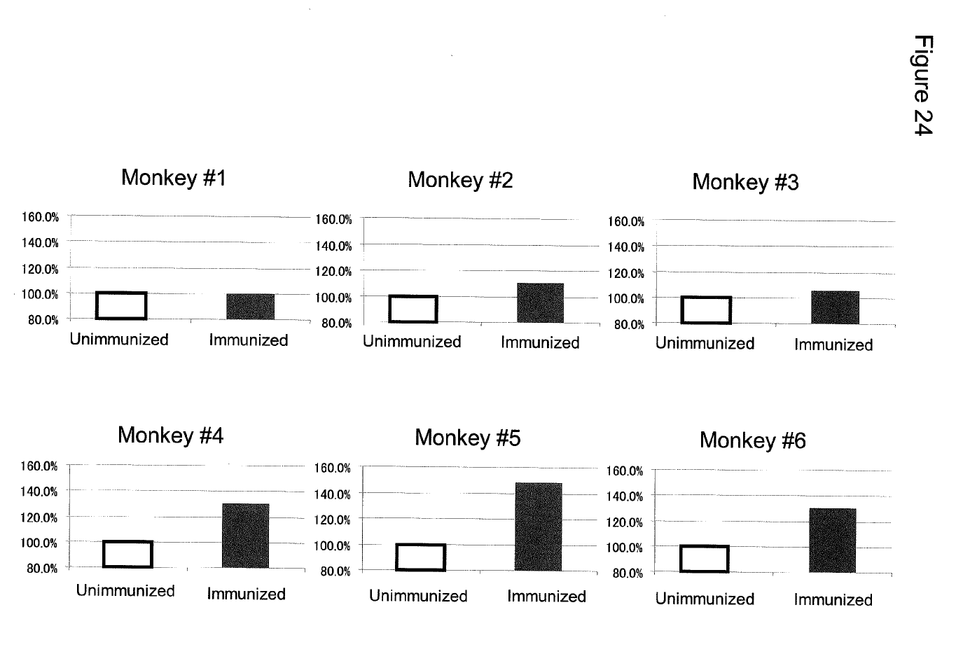

The results obtained with monkeys are shown in FIGS. 23 and 24. FIG. 23 shows the anti-pEAβ3-42 antibody-inducing effect produced by YM3711, and FIG. 24 shows the anti-ADan antibody-inducing effect produced by YM3711. In FIGS. 23 and 24, “Monkeys #1 to #3” show the results in monkeys administered with the existing vaccine (pVAX1/IgL-Aβ1-42-huIgFc), while “Monkeys #4 to #6” show the results in monkeys administered with YM3711.

These results indicated that YM3711 induced anti-pEAβ3-42 antibody and anti-ADan antibody in the monkeys administered therewith. Table 5 below shows the % increase in antibody titer after immunization with YM3711.

TABLE 5
Increase (%) in antibody titer after immunization

| | (%) | | |
|---|---|---|---|
| Anti-pEAβ3-42 antibody | | | |
| | Monkey #1 | Monkey #2 | Monkey #3 |
| Existing vaccine | 24 | 10 | 30 |
| | Monkey #4 | Monkey #5 | Monkey #6 |
| YM3711 | 43 | 50 | 39 |
| Anti-ADan antibody | | | |
| | Monkey #1 | Monkey #2 | Monkey #3 |
| Existing vaccine | 0 | 9 | 4 |
| | Monkey #4 | Monkey #5 | Monkey #6 |
| YM3711 | 31 | 49 | 30 |

As shown in Table 5, the monkeys administered with the existing vaccine (“Monkeys #1 to #3” in Table 5) showed an increase of 10% to 30% for anti-pEAβ3-42 antibody and 0% to 9% for anti-ADan antibody, when compared to before immunization. In contrast, the monkeys administered with YM3711 (“Monkeys #4 to #6” in Table 5) showed an increase of 39% to 50% for anti-pEAβ3-42 antibody and 30% to 49% for anti-ADan antibody. This indicates that YM3711 causes remarkable induction of anti-pEAβ3-42 antibody and anti-ADan antibody in monkeys, in comparison with the existing vaccine.

Further studies were conducted to determine whether anti-IgFc antibody and anti-IL-4 antibody were induced upon YM3711 immunization, there was no induction of these antibodies. The reason is not clear why antibody against repeats of Aβ is induced, whereas no antibody is induced against the IgFc or IL-4 moiety. However, human IgFc and IL-4 molecules are similar to those of monkeys and hence are recognized as self-components by the monkey's body, so that antibodies against these molecules would be difficult to induce. In contrast, Aβ molecules, which are of self origin, abnormally aggregate and hence the body will recognize the aggregate as a foreign substance to cause antibody production.

This finding is important for clinical use of the vaccine in avoiding unwanted immune responses and in enhancing vaccine safety.

In view of the foregoing, the vaccines of the present invention were found not only to induce a wide range of antibodies against various neurotoxic molecules, including predominantly Aβ, in Alzheimer's disease, but also to eliminate Aβ deposition. No DNA vaccine showing such a mechanism of action has been reported in the past.

### INDUSTRIAL APPLICABILITY

The present invention provides DNA vaccines for prevention or treatment of Alzheimer's disease. The vaccines of the present invention remarkably suppress Aβ accumulation and hence are useful as pharmaceutical compositions for treatment or prevention of Alzheimer's disease.

### SEQUENCE LISTING FREE TEXT

SEQ ID NO: 1: synthetic DNA

SEQ ID NO: 2: synthetic peptide

SEQ ID NO: 3: synthetic DNA

SEQ ID NO: 4: synthetic peptide

SEQ ID NO: 5: synthetic DNA

SEQ ID NO: 6: synthetic peptide

SEQ ID NO: 7: synthetic DNA

SEQ ID NO: 8: synthetic peptide

SEQ ID NO: 9: synthetic DNA

SEQ ID NO: 10: synthetic peptide

SEQ ID NO: 11: synthetic DNA

SEQ ID NO: 12: synthetic peptide

SEQ ID NO: 13: synthetic DNA

SEQ ID NO: 14: synthetic peptide

SEQ ID NO: 15: synthetic DNA

SEQ ID NO: 16: synthetic peptide

SEQ ID NO: 21: synthetic DNA

SEQ ID NO: 22: synthetic DNA

SEQ ID NO: 23: synthetic DNA

SEQ ID NO: 24: synthetic DNA

SEQ ID NO: 25: synthetic DNA

SEQ ID NO: 26: synthetic DNA

SEQ ID NO: 27: synthetic DNA

SEQ ID NO: 28: synthetic DNA

SEQ ID NO: 29: synthetic DNA

SEQ ID NO: 30: synthetic DNA

SEQ ID NO: 31: synthetic DNA

1. A recombinant vector, which comprises DNA encoding amyloid p and DNA encoding a Th2 cytokine.

2. The vector according to claim 1, which further comprises DNA encoding an immunoglobulin Fc sequence.

3. The vector according to claim 1 or 2, wherein the DNA encoding amyloid consists of repeats of itself.

4. The vector according to claim 1, wherein the amyloid β is Aβ1-42.

5. The vector according to claim 1, wherein the Th2 cytokine is interleukin-4 or M-CSF.

6. A DNA vaccine for prevention or treatment of Alzheimer's disease, which comprises the recombinant vector according to claim 1.

7. A DNA vaccine for elimination of brain Aβ, which comprises the recombinant vector according to claim 1.

8. An inducer of anti-Aβ antibody, which comprises the recombinant vector according to claim 1.


SEQUENCE LISTING (33 sequences)

SEQ ID NO: 1 - DNA, 129 bp, Artificial
  synthetic DNA
gatgcagaattccgacatgactcaggatatgaagttcatcatcaaaaattggtgttctttgcagaagatgtgggttcaaacaaaggtgcaatcattggactcatggtgggcggtgttgtcatagcgaca

SEQ ID NO: 2 - protein, 43 aa, Artificial
  Synthetic Construct
DAEFRHDSGYEVHHQKLVFFAEDVGSNKGAIIGLMVGGVVIAT

SEQ ID NO: 3 - DNA, 60 bp, Artificial
  synthetic DNA
gatgcagaattccgacatgactcaggatatgaagttcatcatcaaaaattggtgttcttt

SEQ ID NO: 4 - protein, 20 aa, Artificial
  Synthetic Construct
DAEFRHDSGYEVHHQKLVFF

SEQ ID NO: 5 - DNA, 120 bp, Artificial
  synthetic DNA
gatgcagaattccgacatgactcaggatatgaagttcatcatcaaaaattggtgttctttgcagaagatgtgggttcaaacaaaggtgcaatcattggactcatggtgggcggtgttgtc

SEQ ID NO: 6 - protein, 40 aa, Artificial
  Synthetic Construct
DAEFRHDSGYEVHHQKLVFFAEDVGSNKGAIIGLMVGGVV

SEQ ID NO: 7 - DNA, 126 bp, Artificial
  synthetic DNA
gatgcagaattccgacatgactcaggatatgaagttcatcatcaaaaattggtgttctttgcagaagatgtgggttcaaacaaaggtgcaatcattggactcatggtgggcggtgttgtcatagcg

SEQ ID NO: 8 - protein, 42 aa, Artificial [sequence omitted]

SEQ ID NO: 9 - DNA, 129 bp, Artificial
  synthetic DNA
gatgcagaattcggacatgattcaggatttgaagtccgccatcaaaaactggtgttctttgctgaagatgtgggttcgaacaaaggcgccatcatcggactcatggtgggcggcgttgtcatagcaacc

SEQ ID NO: 10 - protein, 43 aa, Artificial
  Synthetic Construct
DAEFGHDSGFEVRHQKLVFFAEDVGSNKGAIIGLMVGGVVIAT

SEQ ID NO: 11 - DNA, 60 bp, Artificial
  synthetic DNA
gatgcagaattcggacatgattcaggatttgaagtccgccatcaaaaactggtgttcttt

SEQ ID NO: 12 - protein, 20 aa, Artificial
  Synthetic Construct
DAEFGHDSGFEVRHQKLVFF

SEQ ID NO: 13 - DNA, 120 bp, Artificial
  synthetic DNA
gatgcagaattcggacatgattcaggatttgaagtccgccatcaaaaactggtgttctttgctgaagatgtgggttcgaacaaaggcgccatcatcggactcatggtgggcggcgttgtc

SEQ ID NO: 14 - protein, 40 aa, Artificial
  Synthetic Construct
DAEFGHDSGFEVRHQKLVFFAEDVGSNKGAIIGLMVGGVV

SEQ ID NO: 15 - DNA, 126 bp, Artificial
  synthetic DNA
gatgcagaattcggacatgattcaggatttgaagtccgccatcaaaaactggtgttctttgctgaagatgtgggttcgaacaaaggcgccatcatcggactcatggtgggcggcgttgtcatagca

SEQ ID NO: 16 - protein, 42 aa, Artificial
  Synthetic Construct
DAEFGHDSGFEVRHQKLVFFAEDVGSNKGAIIGLMVGGVVIA

SEQ ID NO: 17 - DNA, 462 bp, Homo sapiens
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 18 - DNA, 423 bp, Mus musculus
atgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 19 - DNA, 1665 bp, Homo sapiens
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtag

SEQ ID NO: 20 - DNA, 1659 bp, Mus musculus
atgaccgcgcggggcgccgcggggcgctgcccttcttcgacatggctgggctcccggctgctgctggtctgtctcctcatgagcaggagtattgccaaggaggtgtcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaaaggactggaacatttttaccaagaactgcaacaacagctttgctaagtgctctagccgagatgtggtgaccaagcctgattgcaactgcctgtaccctaaagccacccctagcagtgacccggcctctgcctcccctcaccagccccccgccccctccatggcccctctggctggcttggcttgggatgattctcagaggacagagggcagctccctcttgcccagtgagcttccccttcgcatagaggacccaggcagtgccaagcagcgaccacccaggagtacctgccagaccctcgagtcaacagagcaaccaaaccatggggacagactcactgaggactcacaacctcatccttctgcgggggggcccgtccctggggtggaagacattcttgaatcttcactgggcactaactgggtcctagaagaagcttctggagaggctagtgagggatttttgacccaggaagcaaagttttccccctccacgcctgtagggggcagcatccaggcagagactgacagacccagggccctctcagcatctccattccctaaatcaacagaggaccaaaagccagtggatataacagacaggccgttgacagaggtgaaccctatgagacccattggccagacacagaataatactcctgagaagactgatggtacatccacgctgcgtgaagaccaccaggagccaggctctccccatattgcgacaccgaatccccaacgagtcagcaactcagccacccccgttgctcagttactgcttcccaaaagccactcttggggcattgtgctgccccttggggagcttgagggcaagagaagtaccagggatcgaaggagccccgcagagctggaaggaggatcagcaagtgagggggcagccaggcctgtggcccgttttaattccattcctttgactgacacaggccatgtggagcagcatgagggatcctctgacccccagatccctgagtctgtcttccacctgctggtgccgggcatcatcctagtcttgctgactgttgggggcctcctgttctacaagtggaagtggaggagccatcgagaccctcagacattggattcttctgtggggcgaccagaggacagctccctgacccaggatgaggacagacaggtggaactgccagtatag

SEQ ID NO: 21 - DNA, 693 bp, Artificial
  synthetic DNA
gccaccatggagacagacacactcctgctatgggtactgctgctctgggttccaggttccactggtgacgcggccctcgaggatgcagaattccgacatgactcaggatatgaagttcatcatcaaaaattggtgttctttgcagaagatgtgggttcaaacaaaggtgcaatcattggactcatggtgggcggtgttgtcatagcgggtacctcttctggtggtggtggtatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 22 - DNA, 654 bp, Artificial
  synthetic DNA
gccaccatggagacagacacactcctgctatgggtactgctgctctgggttccaggttccactggtgacgcggccctcgaggatgcagaattccgacatgactcaggatatgaagttcatcatcaaaaattggtgttctttgcagaagatgtgggttcaaacaaaggtgcaatcattggactcatggtgggcggtgttgtcatagcgggtacctcttctggtggtggtggtatgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 23 - DNA, 2527 bp, Artificial
  synthetic DNA
gccaccatggagacagacacactcctgctatgggtactgctgctctgggttccaggttccactggtgacgcggccctcgaggatgcagaattccgacatgactcaggatatgaagttcatcatcaaaaattggtgttctttgcagaagatgtgggttcaaacaaaggtgcaatcattggactcatggtgggcggtgttgtcatagcgtgagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccctagtcctgcaggtttaaacgaattcgcccttgaggatccaccatgaccgcgcggggcgccgcggggcgctgcccttcttcgacatggctgggctcccggctgctgctggtctgtctcctcatgagcaggagtattgccaaggaggtgtcagaacactgtagccacatgattgggaatggacacctgaaggtcctgcagcagttgatcgacagtcaaatggagacttcatgccagattgcctttgaatttgtagaccaggaacagctggatgatcctgtttgctacctaaagaaggccttttttctggtacaagacataatagatgagaccatgcgctttaaagacaacacccccaatgctaacgccaccgagaggctccaggaactctccaataacctgaacagctgcttcaccaaggactatgaggagcagaacaaggcctgtgtccgaactttccatgagactcctctccagctgctggagaagatcaagaacttctttaatgaaacaaagaatctccttgaaaaggactggaacatttttaccaagaactgcaacaacagctttgctaagtgctctagccgagatgtggtgaccaagcctgattgcaactgcctgtaccctaaagccacccctagcagtgacccggcctctgcctcccctcaccagccccccgccccctccatggcccctctggctggcttggcttgggatgattctcagaggacagagggcagctccctcttgcccagtgagcttccccttcgcatagaggacccaggcagtgccaagcagcgaccacccaggagtacctgccagaccctcgagtcaacagagcaaccaaaccatggggacagactcactgaggactcacaacctcatccttctgcgggggggcccgtccctggggtggaagacattcttgaatcttcactgggcactaactgggtcctagaagaagcttctggagaggctagtgagggatttttgacccaggaagcaaagttttccccctccacgcctgtagggggcagcatccaggcagagactgacagacccagggccctctcagcatctccattccctaaatcaacagaggaccaaaagccagtggatataacagacaggccgttgacagaggtgaaccctatgagacccattggccagacacagaataatactcctgagaagactgatggtacatccacgctgcgtgaagaccaccaggagccaggctctccccatattgcgacaccgaatccccaacgagtcagcaactcagccacccccgttgctcagttactgcttcccaaaagccactcttggggcattgtgctgccccttggggagcttgagggcaagagaagtaccagggatcgaaggagccccgcagagctggaaggaggatcagcaagtgagggggcagccaggcctgtggcccgttttaattccattcctttgactgacacaggccatgtggagcagcatgagggatcctctgacccccagatccctgagtctgtcttccacctgctggtgccgggcatcatcctagtcttgctgactgttgggggcctcctgttctacaagtggaagtggaggagccatcgaaaccctcagacattggattcttctgtggggcgaccagaggacagctccctgacccaggatgaggacagacaggtggaactgccagtatag

SEQ ID NO: 24 - DNA, 18 bp, Artificial
  synthetic DNA
tcttctggtggtggtggt

SEQ ID NO: 25 - DNA, 1323 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggtaccgagcccaagagcagcgacaagacccacaccagccctccaagccctgcccctgagctgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagcctcgggagcctcaggtgtacaccctgccccccagcagggacgagctgaccaagaaccaggtgtccctgacctgcctggtcaagggcttctaccccagcgatatcgccgtggagtgggagagcaacggccagcccgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccctggcaaggtcgacagcagcggaggcggaggccacaagtgcgacatcaccctgcaggaaatcatcaagaccctgaacagcctgaccgagcagaaaaccctgtgcaccgagctgaccgtgaccgacatcttcgccgccagcaagaacaccaccgagaaagagacattctgcagagccgccaccgtgctgcggcagttctacagccaccacgagaaggacaccagatgtctgggcgccaccgcccagcagttccaccggcacaagcagctgatccggttcctgaagcggctggacagaaatctgtggggcctggccggcctgaacagctgccccgtgaaagaggccaaccagagcaccctggaaaactttctggaacggctcaagaccatcatgcgggagaagtacagcaagtgcagcagctga

SEQ ID NO: 26 - DNA, 1311 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggtaccgagccccgggtgcccatcacccagaaccccagccccccactgaaagagagccccccctctgccgctcctgatctgctgggcggacccagcgtgttcatcttcccacccaagatcaaggacgtgctgatgatcagcctgagccccatggtgaccagcgtggtggtggacgtgtccgaggacgaccccgacgtgcagatcagttggttcgtgaacaacgtggaggtgcacaccgcccagacccagacccaccgggaggactacaacagcaccctgagagtggtgtccgccctgcccatccagcaccaggactggatgagcggcaaagaattcaagagcaaagtgaacaaccgggccctgcccagccccatcgagaaaaccatcagcaagcccagaggccctgtgcgggctcctcaggtgtacgtgctgcccccacccgccgaggaaatgaccaagaaagagttcagcctgaccagcatgatcaccggctttctgcccgccgagatcgccgtggactggaccagcaacggccggaccgagcagaactacaagaacaccgccaccgtgctggacagcgacggcagctacttcatgtacagcaagctgcgggtgcagaagtccacctgggagagaggcagcctgttcgccagcagcgtggtgcacgagggcctgcacaaccacctgaccaccaagaccatcagccggtccctgggagtcgacagcagcggaggcggaggccacatccacggctgcgacaagaaccacctgagagagatcatcggcatcctgaacgaagtgaccggcgagggcaccccctgtaccgagatggacgtgcccaacgtgctgaccgccaccaagaacaccaccgagagcgagctggtgtgccgggccagcaaggtgctgcggatcttctacctgaagcacggcaagaccccctgcctgaagaaaaacagcagcgtgctgatggaactgcagcggctgttccgggccttccggtgcctggacagcagcatcagctgcaccatgaacgagagcaagagcaccagcctgaaggactttctggaaagcctgaagagtatcatgcagatggactacagctga

SEQ ID NO: 27 - DNA, 15 bp, Artificial
  synthetic DNA
ggtggcggtggctcg

SEQ ID NO: 28 - DNA, 1746 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggcggaggcggctccgatgccgagttccggcacgattccggctatgaagtccaccatcagaagctcgtcttttttgccgaggatgtggggtctaacaaaggggccattattgggctcatggtcgggggcgtcgtgattgctggcggcggaggcagcgacgctgagtttcgccacgactccggatacgaagtgcatcaccagaagctggtctttttcgctgaagatgtcggcagtaacaagggggctattattggtctgatggtcggcggagtcgtgatcgcagggggagggggaagcgacgccgaattcaggcatgactctggatatgaggtccaccaccagaaactcgtgttttttgctgaggacgtcggctcaaacaaaggcgctatcattggactcatggtcggaggcgtggtcattgcaggtaccgagcccaagagcagcgacaagacccacaccagccctccaagccctgcccctgagctgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagcctcgggagcctcaggtgtacaccctgccccccagcagggacgagctgaccaagaaccaggtgtccctgacctgcctggtcaagggcttctaccccagcgatatcgccgtggagtgggagagcaacggccagcccgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccctggcaaggtcgacagcagcggaggcggaggccacaagtgcgacatcaccctgcaggaaatcatcaagaccctgaacagcctgaccgagcagaaaaccctgtgcaccgagctgaccgtgaccgacatcttcgccgccagcaagaacaccaccgagaaagagacattctgcagagccgccaccgtgctgcggcagttctacagccaccacgagaaggacaccagatgtctgggcgccaccgcccagcagttccaccggcacaagcagctgatccggttcctgaagcggctggacagaaatctgtggggcctggccggcctgaacagctgccccgtgaaagaggccaaccagagcaccctggaaaactttctggaacggctcaagaccatcatgcgggagaagtacagcaagtgcagcagctga

SEQ ID NO: 29 - DNA, 1734 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggcggaggcggctccgatgccgagttccggcacgattccggctatgaagtccaccatcagaagctcgtcttttttgccgaggatgtggggtctaacaaaggggccattattgggctcatggtcgggggcgtcgtgattgctggcggcggaggcagcgacgctgagtttcgccacgactccggatacgaagtgcatcaccagaagctggtctttttcgctgaagatgtcggcagtaacaagggggctattattggtctgatggtcggcggagtcgtgatcgcagggggagggggaagcgacgccgaattcaggcatgactctggatatgaggtccaccaccagaaactcgtgttttttgctgaggacgtcggctcaaacaaaggcgctatcattggactcatggtcggaggcgtggtcattgcaggtaccgagccccgggtgcccatcacccagaaccccagccccccactgaaagagagccccccctctgccgctcctgatctgctgggcggacccagcgtgttcatcttcccacccaagatcaaggacgtgctgatgatcagcctgagccccatggtgaccagcgtggtggtggacgtgtccgaggacgaccccgacgtgcagatcagttggttcgtgaacaacgtggaggtgcacaccgcccagacccagacccaccgggaggactacaacagcaccctgagagtggtgtccgccctgcccatccagcaccaggactggatgagcggcaaagaattcaagagcaaagtgaacaaccgggccctgcccagccccatcgagaaaaccatcagcaagcccagaggccctgtgcgggctcctcaggtgtacgtgctgcccccacccgccgaggaaatgaccaagaaagagttcagcctgaccagcatgatcaccggctttctgcccgccgagatcgccgtggactggaccagcaacggccggaccgagcagaactacaagaacaccgccaccgtgctggacagcgacggcagctacttcatgtacagcaagctgcgggtgcagaagtccacctgggagagaggcagcctgttcgccagcagcgtggtgcacgagggcctgcacaaccacctgaccaccaagaccatcagccggtccctgggagtcgacagcagcggaggcggaggccacatccacggctgcgacaagaaccacctgagagagatcatcggcatcctgaacgaagtgaccggcgagggcaccccctgtaccgagatggacgtgcccaacgtgctgaccgccaccaagaacaccaccgagagcgagctggtgtgccgggccagcaaggtgctgcggatcttctacctgaagcacggcaagaccccctgcctgaagaaaaacagcagcgtgctgatggaactgcagcggctgttccgggccttccggtgcctggacagcagcatcagctgcaccatgaacgagagcaagagcaccagcctgaaggactttctggaaagcctgaagagtatcatgcagatggactacagctga

SEQ ID NO: 30 - DNA, 1335 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggcggaggcggctccgatgccgagttccggcacgattccggctatgaagtccaccatcagaagctcgtcttttttgccgaggatgtggggtctaacaaaggggccattattgggctcatggtcgggggcgtcgtgattgctggcggcggaggcagcgacgctgagtttcgccacgactccggatacgaagtgcatcaccagaagctggtctttttcgctgaagatgtcggcagtaacaagggggctattattggtctgatggtcggcggagtcgtgatcgcagggggagggggaagcgacgccgaattcaggcatgactctggatatgaggtccaccaccagaaactcgtgttttttgctgaggacgtcggctcaaacaaaggcgctatcattggactcatggtcggaggcgtggtcattgcaggtaccgagcccaagagcagcgacaagacccacaccagccctccaagccctgcccctgagctgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagcctcgggagcctcaggtgtacaccctgccccccagcagggacgagctgaccaagaaccaggtgtccctgacctgcctggtcaagggcttctaccccagcgatatcgccgtggagtgggagagcaacggccagcccgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccctggcaagtga

SEQ ID NO: 31 - DNA, 1350 bp, Artificial
  synthetic DNA
gccaccatggaaaccgacaccctgctgctgtgggtgctgctgctctgggtcccaggatctacaggcgacgccgccctcgaggacgccgagttcagacacgacagcggctacgaggtgcaccaccagaaactggtgttcttcgccgaggacgtgggcagcaacaagggcgccatcatcggcctgatggtcggcggagtggtcattgccggcggaggcggctccgatgccgagttccggcacgattccggctatgaagtccaccatcagaagctcgtcttttttgccgaggatgtggggtctaacaaaggggccattattgggctcatggtcgggggcgtcgtgattgctggcggcggaggcagcgacgctgagtttcgccacgactccggatacgaagtgcatcaccagaagctggtctttttcgctgaagatgtcggcagtaacaagggggctattattggtctgatggtcggcggagtcgtgatcgcagggggagggggaagcgacgccgaattcaggcatgactctggatatgaggtccaccaccagaaactcgtgttttttgctgaggacgtcggctcaaacaaaggcgctatcattggactcatggtcggaggcgtggtcattgcaggtaccgagccccgggtgcccatcacccagaaccccagccccccactgaaagagagccccccctctgccgctcctgatctgctgggcggacccagcgtgttcatcttcccacccaagatcaaggacgtgctgatgatcagcctgagccccatggtgaccagcgtggtggtggacgtgtccgaggacgaccccgacgtgcagatcagttggttcgtgaacaacgtggaggtgcacaccgcccagacccagacccaccgggaggactacaacagcaccctgagagtggtgtccgccctgcccatccagcaccaggactggatgagcggcaaagaattcaagagcaaagtgaacaaccgggccctgcccagccccatcgagaaaaccatcagcaagcccagaggccctgtgcgggctcctcaggtgtacgtgctgcccccacccgccgaggaaatgaccaagaaagagttcagcctgaccagcatgatcaccggctttctgcccgccgagatcgccgtggactggaccagcaacggccggaccgagcagaactacaagaacaccgccaccgtgctggacagcgacggcagctacttcatgtacagcaagctgcgggtgcagaagtccacctgggagagaggcagcctgttcgccagcagcgtggtgcacgagggcctgcacaaccacctgaccaccaagaccatcagccggtccctgggatga

SEQ ID NO: 32 - DNA, 696 bp, Homo sapiens
gagcccaagagcagcgacaagacccacaccagccctccaagccctgcccctgagctgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagcctcgggagcctcaggtgtacaccctgccccccagcagggacgagctgaccaagaaccaggtgtccctgacctgcctggtcaagggcttctaccccagcgatatcgccgtggagtgggagagcaacggccagcccgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccctggcaag

SEQ ID NO: 33 - DNA, 711 bp, Mus musculus
gagccccgggtgcccatcacccagaaccccagccccccactgaaagagagccccccctctgccgctcctgatctgctgggcggacccagcgtgttcatcttcccacccaagatcaaggacgtgctgatgatcagcctgagccccatggtgaccagcgtggtggtggacgtgtccgaggacgaccccgacgtgcagatcagttggttcgtgaacaacgtggaggtgcacaccgcccagacccagacccaccgggaggactacaacagcaccctgagagtggtgtccgccctgcccatccagcaccaggactggatgagcggcaaagaattcaagagcaaagtgaacaaccgggccctgcccagccccatcgagaaaaccatcagcaagcccagaggccctgtgcgggctcctcaggtgtacgtgctgcccccacccgccgaggaaatgaccaagaaagagttcagcctgaccagcatgatcaccggctttctgcccgccgagatcgccgtggactggaccagcaacggccggaccgagcagaactacaagaacaccgccaccgtgctggacagcgacggcagctacttcatgtacagcaagctgcgggtgcagaagtccacctgggagagaggcagcctgttcgccagcagcgtggtgcacgagggcctgcacaaccacctgaccaccaagaccatcagccggtccctggga